(19) United States
(12) Patent Application Publication
(10) Pub. No.: US 2014/0227335 A1
(43) Pub. Date: Aug. 14, 2014

(54) VIRUCIDAL SMALL MOLECULE AND USES THEREOF

(71) Applicants: Zhilei Chen, College Station, TX (US); Karrupiah Chockalingam, College Station, TX (US); Ana M. Chamoun-Emanuelli, College Station, TX (US); Rudo Simeon, College Station, TX (US); Michael Bobardt, La Jolla, CA (US); Philippe Gallay, La Jolla, CA (US)

(72) Inventors: Zhilei Chen, College Station, TX (US); Karrupiah Chockalingam, College Station, TX (US); Ana M. Chamoun-Emanuelli, College Station, TX (US); Rudo Simeon, College Station, TX (US); Michael Bobardt, La Jolla, CA (US); Philippe Gallay, La Jolla, CA (US)

(21) Appl. No.: 14/023,434

(22) Filed: Sep. 10, 2013

Related U.S. Application Data
(60) Provisional application No. 61/699,104, filed on Sep. 10, 2012.

(51) Int. Cl. A61K 31/542; A61K 45/06

(52) U.S. Cl. CPC A61K 31/542; A61K 45/06
USPC 424/422; 514/224.5; 424/85.4; 435/238

(57) ABSTRACT

In some embodiments the present disclosure provides a method of inactivating a RNA virus of the family retroviridae in a biological sample. Such a method comprises contacting the biological sample with an effective amount of an antiviral composition comprising PD 404,182. In an embodiment of the present disclosure the RNA virus is selected from the group consisting of HIV pseudotyped lentiviruses, primary human immunodeficiency virus-1 isolates (HIV-1), human immunodeficiency virus-2 (HIV-2), and simian immunodeficiency virus (SIV). Further embodiments of the present disclosure pertain to a method of treating a subject infected by a RNA virus of the family retroviridae. Another embodiment of the present invention pertains to a method of preventing transmission of a RNA virus of the family retroviridae in a subject in need thereof.

### CROSS-REFERENCE TO RELATED APPLICATIONS

This application claims priority to U.S. Provisional Application No. 61/699,104 filed Sep. 10, 2012. The entirety of the aforementioned application is incorporated herein by reference.

### STATEMENT REGARDING FEDERALLY SPONSORED RESEARCH

This invention was made with government support under NIH grant 1R21AI083965-01. The government has certain rights in the invention.

### BACKGROUND

Human pathogenic viruses that acquire resistance to antiviral agents by rapid evolution in vivo pose a serious health problem with no simple cure. Majority of the existing anti-virals target viral genome encoded structures of these viruses. Such therapeutics often exhibit limited efficacy, because of the ability of the virus to alter the viral genome encoded targets. Additionally, patients co-infected with more than one virus exhibit a higher rate of viral persistence, increased viral load, and higher susceptibility to death, as compared to individuals infected with only a single virus. Current therapies have limited efficacy and foster the development of resistant strains. Therefore, there is a need to develop new anti-viral therapies that treat and prevent infections through new modes of actions that do not necessarily target viral genome encoded structures.

### BRIEF SUMMARY

In some embodiments the present disclosure provides a method of inactivating a RNA virus of the family retroviridae in a biological sample. Such a method comprises contacting the biological sample with an effective amount of an antiviral composition comprising PD 404,182. In an embodiment of the present disclosure the RNA virus is selected from the group consisting of HIV pseudotyped lentiviruses, primary human immunodeficiency virus-1 isolates (HIV-1), human immunodeficiency virus-2 (HIV-2), and simian immunodeficiency virus (SIV). In some embodiments, the inactivation of the virus particle by PD 404,182 is mediated by physical disruption of the virion.

Further embodiments of the present disclosure pertain to a method of treating a subject infected by a RNA virus of the family retroviridae. Such a method comprises administering to the subject a therapeutically or prophylactically effective amount of an antiviral composition comprising PD 404,182 or a pharmaceutically acceptable salt thereof. In an embodiment the method is effective in treating, preventing or reducing a viral infection in a subject infected with HIV. In some embodiments, the subject infected with HIV is co-infected with HCV.

Another embodiment of the present invention pertains to a method of preventing transmission of a RNA virus of the family retroviridae in a subject in need thereof. Such method comprises contacting a mucus membrane of the subject with a microbicide topical formulation comprising an effective amount of PD 404,182 or a pharmaceutically acceptable salt thereof. Specifically, the mucus membrane is that of the cervix or of the rectum. In an embodiment, the RNA virus of the retroviridae family is the HIV pseudotyped lentiviruses, human immunodeficiency virus-1 (HIV-1), human immunodeficiency virus-2 (HIV-2), and simian immunodeficiency virus (SIV). In some embodiments, the RNA virus of the retroviridae family is the human immunodeficiency virus-1 (HIV-1) or the human immunodeficiency virus-2 (HIV-2).

### BRIEF DESCRIPTION OF THE FIGURES

FIGS. 1A-1B show the virucidal activity of PD 404,182 (PD) against HCVcc. Jc1 HCVcc was incubated with PD or 0.5% DMSO at 37° C. for 30 minutes, diluted 1000-fold, and used to infect Huh-7.5 cells. The control sample contains virus and PD of the same final titer/concentrations, but with the virus and PD separately diluted 1000-fold prior to mixing. The infectivity was quantified by measuring the supernatant activity of the Gluc reporter 72 h post infection (FIG. 1A). Inset: chemical structure of PD (FIG. 1A). HCV-infected cells are brown after immunostaining (FIG. 1B).

FIG. 1C shows effect of PD 404,182 (PD) on extracellular VSV-Gpp and HCVcc. VSVGpp (harboring pV1-B, ˜10⁶ TCID₅₀/mL; undiluted) or HCVcc (10⁴ TCID₅₀/mL, 10-fold diluted) was incubated with PD (150 μM in 0.5% DMSO) or 0.5% DMSO in the presence of 7 ng/ml RNase A at 37° C. for 30 min. The viral RNA levels of the virus-PD and virus-DMSO mixtures were quantified by qRT-PCR, while the infectivity of the same mixtures was determined by spinoculation of Huh-7.5 cells and quantification of intracellular viral RNA by qRT-PCR 48 h later. All data are the mean±SD of 2 independent experiments carried out in duplicate.

FIG. 2 shows PD de-stabilizes primary HIV-1 particles. NL4.3 virus (20 ng of p24) was incubated in the presence or absence of 10 μM PD for 30 min at 37° C. and loaded over a sucrose density gradient. Quantification of HIV-1 capsid and RT proteins was conducted by p24 ELISA and exoRT assay, respectively.

FIGS. 3A-3B show PD 404,182 (PD) does not lyse or directly interact with liposomal membranes. The ability of PD (300 μM), the virucidal peptide C5A (10 μM), and solvent DMSO (1%) to permeabilize liposomes entrapping SulfoB was determined by a liposome dye release assay (FIG. 3A). A relative fluorescence intensity of 100 corresponds to SulfoB release resulting from liposome disruption with 0.1% Triton X-100. VSV-Gpp (˜1.7×10⁷ TCIdD₅₀/mL) was diluted 500-fold in fresh complete growth medium and pre-incubated with PD for 30 min at 37° C. in the presence or absence of various concentrations of liposomes (FIG. 3B). The virus/PD/liposome mixtures were then used to spinoculate Huh-7.5 cells and infectivity was quantified by measuring the supernatant activity of the Gluc reporter 48 h post transduction. All data are the mean±SD of 2 independent experiments carried out in triplicate.

FIGS. 4A-4D show the virucidal activity of PD is temperature-, time- and virus dilution-dependent. Undiluted VSV-Gpp (5×10⁵ TCID₅₀/mL) was treated with 300 μM PD, 0.1% Triton X-100 or 1% DMSO in the presence of RNase A for 60 minutes at different temperatures (FIG. 4A) or at 37° C. for different times (FIG. 4B). Viral RNA was isolated thereafter and quantified using qRT-PCR. For virus dilution studies, VSV-Gpp stock (1.7×10⁷ TCID₅₀/mL) was diluted 500-fold in medium comprising different proportions of conditioned and fresh complete media (all containing 10% FBS) or fresh serum-free medium (FIG. 4C), or the flow-through of conditioned medium size-fractionated through membranes with pores of the indicated size, prior to pre-treatment with PD at 37° C. for 30 min and spinoculation of naive Huh-7.5 cells (FIG. 4D). Infectivity was quantified 2 days later by measuring the supernatant activity of the Gluc reporter. All data are the mean±SD of 2 independent experiments carried out in duplicate.

FIGS. 5A-5B show effect of PD on HIV-1 infection. Antiviral effect of PD before, during, and after virus exposure (FIG. 5A). PD (10 μM) or just growth medium (DMSO control) was added to TZM-b1 cells 1, 2, 4, 8 or 16 h before (negative values on the y axis), after (positive values on the y axis) the addition of HIV-1 (R5 JR-CSF) (1 ng of p24) or together (time zero) with the virus. Infection was quantified 48 h later via measurement of β-galactosidase activity. DC (10⁵ cells) were incubated for 2 h at 37° C. with wild-type NL4.3-eGFP (X4 virus) and NL4.3-BaL-eGFP (R5 virus) viruses or with the pseudotyped NL4.3i1Env-eGFP/gp160×4 Env virus (25 ng of p24). PD (10 μM) or control DMSO medium was added 2 h later (FIG. 5B). DC were washed 2 h after adding PD, Jurkat T cells (100,000 cells) were added for 3 days, and the, percentage of infected Jurkat T cells (GFP+) was analyzed by flow cytometry. Error bars represent standard errors of duplicates from 2 independent experiments.

FIG. 6 shows PD inhibits infection by different HIV pseudotyped lentiviruses. Lentiviruses (harboring pV1-Gluc provirus) pseudotyped with envelope proteins from Sindbis virus (SINVpp), murine leukemia virus (MLVpp), human immunodeficiency virus (HIVpp) and vesicular stomatitis virus (VSV-Gpp) were incubated with PD at 37° C. for 30 min, and used to spinoculate BHK-J (SINVpp), Huh-7.5 (MLVpp, VSV-Gpp) and TZM-b1 (HIVpp) cells at 4° C. Cells were washed 4 times with fresh medium to remove any unbound viruses and compound, and incubated at 37° C./5% CO2. Viral infectivity was determined by measuring the supernatant activity of the Gluc reporter 48 h later. Due to differences in specific infectivity, SINVpp, MLVpp, HIVpp and VSV-Gpp virus stocks were diluted 5-, 50-, 10- and 100-fold, respectively, with fresh complete growth medium prior to compound treatment. The different virus dilutions ensured a similar final titer of the different viruses, as judged by the similar supernatant activities of the Gluc reporter after dilution. The error bars represent the mean SD± of 2 independent experiments performed in duplicate.

FIGS. 7A-7B show effect of PD on HCVcc virion integrity and attachment to cells. PD only weakly disrupts HCVcc (FIG. 7A). HCVcc (10⁴ TCID₅₀/mL) was incubated with PD (300 μM), Triton X-100 (0.1%) or 1% DMSO in the presence of 7 ng/mL RNase A at 37° C. for 90 min. Isolation and quantification of viral RNA was carried out. HCVcc cell attachment assay (FIG. 7B). Jc1 HCVcc was partially clarified by four serial passages through a 300 kDa cutoff ultra-filtration membrane (Pall Life Sciences, Port Washington, N.Y.). With each passage through the centrifugal unit, the retained virus was diluted in PBS prior to the next passage. HCVcc (10⁴ TCID₅₀/ml) was pre-incubated with either freshly prepared heparin (1000 μg/ml; positive attachment inhibitor control) from porcine intestinal mucosa (Sigma, St. Louis, Mo.), 300 μM PD, or 1% DMSO at 37° C. for 90 minutes under low-serum (<1%) conditions. In preparation for virus addition, Huh-7.5 cells seeded one day earlier at 3×10⁵ cells/well, in 24-well plates, were chilled on ice for 5 minutes. After aspirating the existing medium from the cells, 50 μl of the pre-treated HCVcc was added per well and the virus/cell mixture was incubated at 4° C. for an additional 3 h. Cells were subsequently washed 5 times with complete growth medium and incubated at 37° C./5% CO₂ for an additional 2 h. Total RNA was harvested from the cells using the RNeasy Mini Kit (Qiagen, Valencia, Calif.). RNA levels of cell-bound HCVcc were determined via TaqMan qRT-PCR. Note: The observed slight decrease in attachment of PD-treated HCVcc relative to DMSO-treated virus is likely due to virion lysis (see (FIG. 7A)). The error bars represent the mean SD± of 2 independent experiments done in duplicate.

FIGS. 8A-8B show effect of PD on SINV and DenV. Sindbis virus was produced in cell culture by electroporation of BHK-J cells with in vitro-transcribed viral RNA (FIG. 8A). Briefly, plasmid carrying the genome of SINV (Toto1101) was linearized by digestion with XhoI and 1 μg of the linearized plasmid was used as a template for run-off transcription with SP6 RNA polymerase (Ampliscribe SP6 High-Yield Transcription Kit, Epicentre, Madison, Wis.). BHK-J cells were trypsinized, resuspended in cold DPBS to 2.8×10⁷ cells/ml and 400 μl of this cell suspension was electroporated with 3 μg of in vitro-transcribed viral RNA using an ECM 830 electroporator (Harvard Apparatus, Holliston, Mass.) using the following settings: 750 V, 5 pulses, 99 us pulse length, 1 second intervals. Virus-containing supernatant was collected 24 h post electroporation and stored at −80° C. Virus titer was determined on BHK-J cells with 10-fold serial dilutions of sample, and then plaques were visually enumerated after crystal violet staining, as previously described. For determination of the inhibitory effect of PD 404,182, cell culture-produced SINV was diluted 1000-fold in complete growth medium to 10⁵ pfu/ml and pre-incubated with 300 μM PD 404,182 or 1% DMSO at 37° C. for 1 h. Pre-incubated virus was diluted a further 2000-fold and used to inoculate BHK-J cells for enumeration of plaques. Serotype 2 New Guinea C strain Dengue virus was propagated in Vero cells (FIG. 8B). Dengue virus serially diluted in complete medium containing 10% FBS was incubated with PD (10, 100 or 300 μM) or DMSO at 37° C. for 30 min and used to infect Vero cells in a standard plaque assay. Briefly, Vero cells were seeded in 24-well plates at 10⁵ cells/well and inoculated with 100 μL PD- or mock-treated Dengue virus at 37° C. for 1 h. After removal of the inoculum, these cells were overlayed with 1 ml of culture medium containing 0.5% methyl cellulous. Five days later, the cells were fixed and stained with crystal violet to visualize plaques. The error bars represent the mean±SD of 2 independent experiments done in duplicate.

FIGS. 9A-9C show effect of prolonged incubation of PD with liposomes. No significant increase in fluorescence intensity was observed even after prolonged incubation of liposome with PD (FIG. 9A). The virocidal activity of PD is not attenuated by the presence of liposomes (FIGS. 9B-9C). Liposomes composed of 70 mg POPC and 30 mg cholesterol (FIG. 9B) or 12 mg POPC, 33 mg SM, 5 mg PE, 19 mg pl-PE (FIG. 9C), 30 mg cholesterol and 1 mg POPS (the same composition as HIV per 100 mg were incubated with PD and VSV-Gpp as described in FIG. 3B. The error bars represent the mean±SD of 2 independent experiments done in duplicate. (POPC: 1-Palmitoyl-2-oleoyl-sn-Glycero-3-Phosphocholine; Cho: cholesterol; SM: sphingomyelin; PE: 1-palmitoyl-2-oleoyl-sn-glycero-3-phosphatidylethanolamine; POPS: 1-palmitoyl-2-oleoyl-sn-glycero-3-phospho-L-serine; pl-PE: 1-alkenyl, 2-acylglycerophosphoethanolamine (Avanti Polar Lipids, Inc., Alabaster, Ala.); DPPC: 1,2-dipalmitoyl-sn-glycero-3-phosphocholine (DPPC) (Fisher Scientific, Pittsburg Pa.).

FIGS. 10A-10 B show the antiviral potency of PD against VSV-Gpp and HIVpp is virus dilution-dependent. VSV-Gpp (FIG. 10A) and HIVpp (FIG. 10B) (both harboring pV1-Gluc provirus) diluted in fresh complete growth medium and treated with PD at 37° C. for 30 min were used to spinoculate Huh-7.5 and TZM-b1 cells, respectively. The titers for 5-fold diluted VSV-Gpp and undiluted HIVpp were 3.4×10⁶ and 2.3×10⁴ TCID₅₀/mL, respectively. The error bars represent the mean±SD of 2 independent experiments done in duplicate.

FIGS. 11A-11B show cytotoxicity of PD 404,182 on different human cell lines. The cytotoxicity of PD was determined in the human cell lines HepG2 (hepatoma), HCT-8 (colon cancer), Huh-7 (hepatoma), Huh-7.5 (hepatoma), TZM (cervical cancer), PC3 (prostate cancer), and 293T (embryonic kidney). Cells were seeded in 96-well flat bottom tissue culture plates at 1.8×10⁴-3.2×10⁴ cells per well, where cell lines that divide faster were seeded at lower densities. After plating, cell culture supernatants were replaced with PD-containing medium, and cells were incubated at 37° C./5% CO₂. At 12 h intervals post initial treatment with compound, cell culture supernatants were removed and replaced with freshly prepared PD diluted in complete growth medium. At 24 h (FIG. 11A) and 48 h (FIG. 11B) post initial treatment with PD, cell viability was determined using Cell Titer-Glo reagent. Cell culture supernatants were removed and replaced with 50 μL Cell Titer-Glo reagent diluted 1:10 in ddH2O. Microplates were then gently vortexed for 2 min and incubated at room temperature for an additional 8 min. Ten microliters of sample from each well was transferred to a white 96-well plate (Corning), and luminescence was measured in a Berthold Tristar LB 941 luminometer for 0.1 s. The error bars represent the mean±SD of 2 independent experiments done in duplicate.

FIG. 12 shows PD exhibits minimum toxicity against primary human cells. Increasing concentrations were incubated with primary CD4⁺ T-lymphocytes, macrophages and dendritic cells at 37° C. and the amounts of lactate dehydrogenase (LDH) present in the culture media were quantified after 0, 7 and 14 days. Error bars represent the standard deviation from two independent experiments.

FIGS. 13A-13B show PD is stable and fully active at acidic pH and in cervical fluid. PD (30 μM) or DMSO (10%) were incubated at 37° C. for 0, 24 or 48 h in (FIG. 13A) DPBS buffered at pH 4, 6, 8 or 10, or (FIG. 13B) 20% cervical fluid (diluent was DPBS). These PD samples were then diluted to the desired concentration in complete growth medium containing VSV-Gpp (viral supernatant diluted 500-fold), and the PD/virus mixtures were incubated at 37° C. for 30 min and used to inoculate naïve Huh-7.5 cells at 4° C. for 2 h prior to incubation at 37° C./5% CO2. The infectivity (virus entry into cells) was quantified by measuring the supernatant Gluc reporter activity 48 h post infection. Values and error bars represent the mean and standard deviation, respectively, of three independent experiments. Statistical significance was determined by Student's t test (*, P<0.01).

FIGS. 14A-14B show the long-term stability of PD. PD (5 μM) or DMSO (1.67%) were diluted in (FIG. 14A) pH-adjusted DPBS (pH 4 or 7) or (FIG. 14B) 1.5% HEC in DPBS (pH 4) and stored at the indicated temperature for 8 weeks. An aliquot was removed each week, diluted to the desired concentration in complete growth media containing VSV-Gpp (500-fold diluted), incubated at 37° C. for 30 min, and used to infect naïve Huh-7.5 cells at 4° C. for 2 h prior to incubation at 37° C./5% CO2. The viral infectivity was quantified by measuring the supernatant Gluc reporter activity 48 h post infection. Error bars represent the standard deviation of duplicate samples. Statistical significance was determined by Student's t-test (*, P<0.05).

FIG. 15 shows PD does not foster the emergence of escape mutants. HIV-1 (1 ng of p24 of NL4.3) was added to TZM-b1 cells (1×106 cells). Fifteen minutes later, an aliquot of supernatant was collected for viral input normalization, and PD was added to cells at the indicated concentrations. Cells were then split every two days for a period of 60 days. Fresh PD was added at each passage to maintain the same concentration throughout the 60 days. Before each passage an aliquot of supernatant was collected to determine amounts of virus in cell

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Unless defined otherwise, all technical and scientific terms used herein have the meaning commonly understood by a person skilled in the art to which the invention belongs. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} ed., R. Reigers et al. (eds.), Springer Verlag (1991); and Hale & Marham, The Harper Collins Dictionary of Biology (1991).

The following definitions are provided for specific terms which are used in the following written description.

The term “treating” or “treatment” as used herein is meant to refer to the administration of a compound or composition according to the present invention to alleviate or eliminate symptoms of the viral infection in the subject and/or to reduce viral load in the subject. In certain examples treating is meant to refer to alleviating or eliminating symptoms of HCV or HIV or both, and/or to reduce viral load in the subject being treated.

Reference herein to “therapeutic” and “prophylactic” is to be considered in their broadest contexts. The term “therapeutic” does not necessarily imply that a mammal is treated until total recovery. Similarly, “prophylactic” does not necessarily mean that the subject will not eventually contract a disease condition. Accordingly, therapy and prophylaxis include amelioration of the symptoms of a particular condition or preventing or otherwise reducing the risk of developing a particular condition. The term “prophylaxis” may be considered as reducing the severity of onset of a particular condition. Therapy may also reduce the severity of an existing condition or the frequency of acute attacks.

As used herein, the term “Subject” includes animals and humans requiring intervention or manipulation due to a disease state, treatment regimen or experimental design.

Since its discovery in humans in 1981, HIV, the causative agent of AIDS, has infected over 60 million people worldwide and caused more than 25 million deaths. For HIV, there are currently over 20 approved antiretroviral drugs, forming the basis of highly active antiretroviral therapy (HAART). Although highly active antiretroviral therapy (HAART) can significantly reduce viral load and prolong patients' life expectancy, these therapies are not curative. Worldwide, nearly half of all individuals living with HIV are women, most of whom acquire the virus after sexual intercourse with HIV-positive men. As receptive partners, women are twice as likely, than their male counterparts to acquire HIV during sex. Despite the knowledge of effective prevention strategies, such as the “ABC” approach (abstinence, be faithful and use of condoms), the rate of HIV transmission remains high in developing countries. Moreover, many women cannot reliably negotiate safe sex practices, leaving them vulnerable to sexually transmitted infections. Thus, the development of a safe, effective and acceptable topical microbicide capable of retarding or preventing the sexual transmission of HIV could empower women to take personal responsibility to prevent HIV acquisition from their infected partners.

Topical microbicides are agents able to inhibit the transmission of viral infections when applied to the vagina, penis and/or lower gastrointestinal (GI) tract via the rectum. An ideal anti-HIV microbicide should fulfill most or all of the following criteria: a) inhibit transmission of wild type and drug-resistant virus; b) stability and potency in seminal fluids and vaginal secretions; c) absence of toxicity to the vaginal epithelium and commensal bacteria flora; d) ability to interfere with multiple transmission modes (e.g. as cell-free vs. cell-associated virus) given unknowns in the exact mode of HIV transmission in vivo; e) possess a high genetic barrier to resistance development; and f) preferably act through a distinct mode of action from existing therapeutics. The last consideration derives from the presence of rare pre-existing drug-resistant viral variants, as well as drug-resistant HIV variants from patients who underwent previous anti-retroviral treatment, that can bypass the microbicidal barrier and transmit to target cells. Most current anti-HIV microbicide candidates in clinical trials are formulated based on existing anti-retroviral drugs and target well-studied viral proteins such as HIV protease (PR), reverse transcriptase (RT) and HIV envelope protein (Env).

To date, four major types of vaginal HIV microbicides have been developed with varying degrees of clinical success: surfactants, entry inhibitors, vaginal milieu protectors and reverse transcriptase inhibitors. Surfactants non-specifically disrupt membranes and were the first molecules to enter clinical trials as candidate HIV microbicides. However, these surfactants were found to be toxic to the cervico-vaginal mucosa and resulted in an increased rate of HIV infection in Phase III clinical trials. Entry inhibitors prevent HIV from binding to or entering cells and encompass a wide range of molecules, including CCR5 inhibitors and fusion inhibitors. Many polyanions have also been developed to inhibit HIV entry and some have been extensively tested in Phase III trials, including PRO2000, cellulose sulfate, and Carraguard. However, most of these candidates have failed to show in vivo efficacy in preventing HIV transmission, partly due to the complexity of the mucosal environment as well as the interference of semen.

Vaginal milieu protectors are designed to maintain or enhance the protective acidic pH of the vaginal environment through the use of strong buffering agents, such as Carbopol 974, or genetically engineered Lactobacilli. An agent that is being considered for HIV microbicidal applications in clinical trials, tenofovir, is a nucleotide analogue that inhibits the reverse transcriptase of HIV. A 1% tenofovir gel applied before and after sexual intercourse was 39% effective overall in preventing HIV infection in women, and 54% effective among highly adherent users of the gel. These data are encouraging, but nevertheless show that the efficacy of tenofovir microbicidal therapy alone is limited.

The recently FDA-approved anti-HIV prophylactic therapeutic, Truvada®, comprises two nucleoside analogs, tenofovir and emtricitabine. Truvada offered a 44% reduction in HIV transmission during initial clinical trials. However, since both tenofovir and emtricitabine are currently used in the clinic for HIV treatment as part of HAART drug cocktail, concerns were raised about the potential for the spread of drug-resistant variants when the drug is used by individuals with unknown or positive HIV status. This issue becomes more significant when the drug is used on a large scale, generating an extra incentive to identify new and specific anti-HIV microbicidal compounds with unique modes of action. In addition, a recently completed comprehensive HIV prevention trial among African women known as VOICE (Vaginal and Oral Interventions to Control the Epidemic) involving tenofovir failed to provide protection against HIV, underscoring the need for additional HIV-prevention options that incentivize patient usage and adherence. Thus, despite the availability of this large repertoire of anti-HIV drugs, drug-resistant mutant strains of HIV still emerge over time.

Approximately 4-5 million HIV patients are co-infected with HCV. HIV patients co-infected with HCV tend to exhibit a higher rate of viral persistence, increased viral load, and higher susceptibility to death compared to individuals infected with only one of these viruses. For HCV, the current interferon/ribavirin combination therapy exhibits limited efficacy and the two recently approved small-molecule drugs, both serine protease inhibitors—telaprevir and boceprevir—foster the development of resistant viral strains within days when administered alone.

Antiviral molecules targeting critical virus structural elements tend to be effective against several viruses and do not usually foster the emergence of drug-resistant viral isolates. One group of molecules inhibits virus-cell fusion by inducing positive membrane curvature, thus increasing the activation energy barrier for fusion with cell membranes. These molecules, which include rigid amphipathic fusion inhibitors (RAFIs) and lysophosphatidylcholine, tend to have large hydrophilic heads and hydrophobic tails. LJ001, a recently discovered broad-spectrum small-molecule antiviral, inhibits the fusogenic activity of enveloped viruses by intercalating into the lipid membrane while leaving virion particles grossly intact. Alkylated porphyrins exhibit strong antiviral activity against several enveloped viruses through an unknown mechanism, perhaps by interfering with specific structures on the virus surface. Amphipathic peptides derived from HCV NS5A protein were shown to physically disrupt virions and were active against a variety of enveloped viruses. Another approach to interfering with membrane elements required for virus infection is to target exposed anionic phospholipids widely expressed on infected host cells and viral envelopes, as was done with Bavituximab, a chimeric antibody which rescues mice from Pichinde virus and mouse cytomegalovirus infection. However, none of these agents has been approved for human use to date. Both LJ001 and RAFIs target lipid membrane including host cellular membrane, which may lead to undesirable toxicity. C5A and Bavituximab are all protein based therapeutic which have short in vivo stability and are expensive to manufacture.

Thus, there is an urgent need to develop anti-virals with high antiviral potency and low cytotoxicity, combined with a unique mode of action to combat infections by these viruses.

The present disclosure pertains to a small molecule PD 404,182 (PD), a colorless, odorless synthetic compound, that has potent antiviral activity against several primary isolates of human immunodeficiency virus (HIV), and Simian immunodeficiency virus (SIV), as well as HIV pseudotyped lentiviruses. PD is a known inhibitor of bacterial KDO 8-P synthase and has recently also been demonstrated to affect angiogenesis, and mammalian circadian rhythm. The present disclosure describes novel methods for treating viral infections, based on the targeting of non-envelope protein viral structural components of viruses.

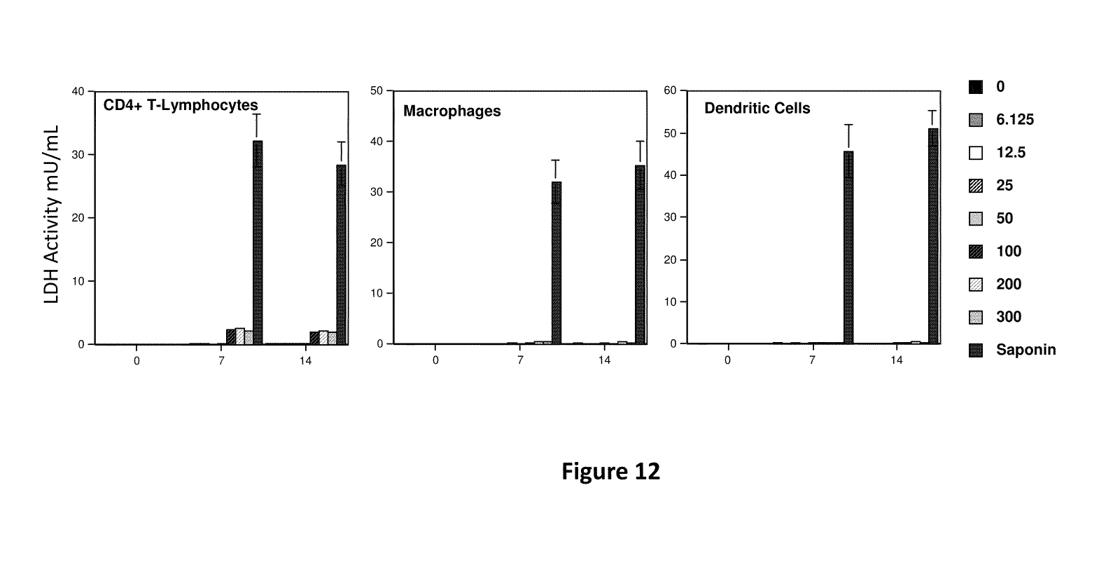

The present disclosure pertains to a synthetic small molecule—PD 404,182 (PD)—that possesses virucidal activity towards retroviruses, including HIV. In contrast to existing anti-HIV microbicidal candidates, PD inactivates HIV via a unique, possibly novel mechanism. PD is the only non-surfactant small molecule reported to physically compromise the integrity of HIV, thus rendering the extracellular virus non-infectious (Table 1 and 2). In addition, PD exhibits low toxicity toward several human cell lines, freshly activated PBMCs (Table 3), primary CD4⁺ T-lymphocytes, macrophages and dendridritic cells (FIG. 12) and normal vaginal flora (Table 2).

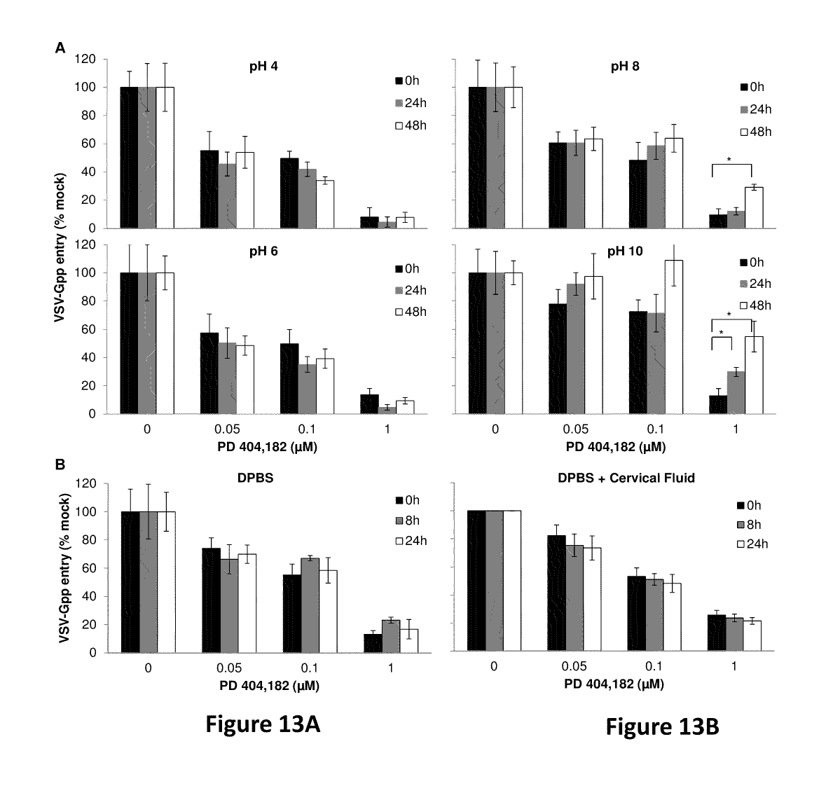

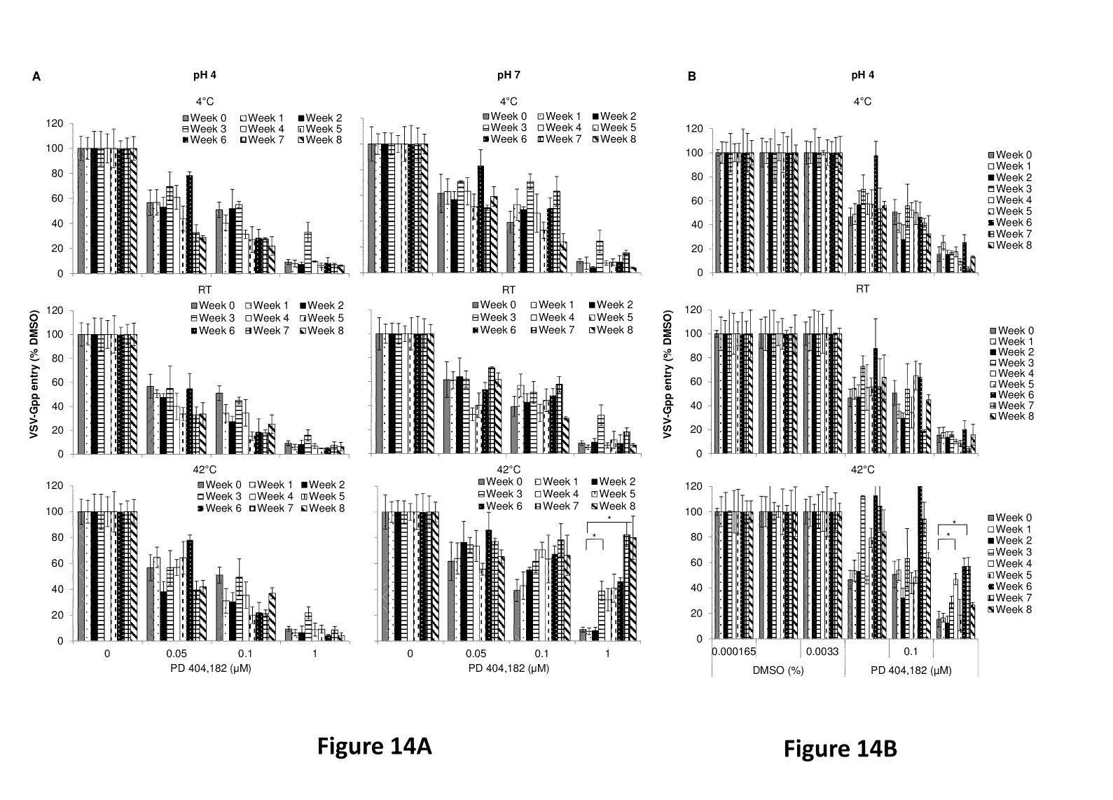

The antiviral potency of PD is not affected by the presence of seminal plasma (Table 1) or exposure to cervical fluid at 37° C. for 24 hours (FIG. 13B), indicating the potential for a once-a-day application of PD for HIV prophylaxis. The very high stability of PD in acidic pH at both room temperature and 42° C., and in neutral pH at room temperature (FIG. 14), indicate that PD can be easily formulated for convenient transportation and storage in developing countries lacking refrigeration facilities. In the present disclosure, Applicants also evaluated the stability of PD when formulated in 1.5% HEC gel. Surprisingly, PD is not stable when formulated in HEC gel at pH 7 (data not shown), but PD formulated in HEC gel at pH 4 retains full potency after 4 weeks at ambient temperature (FIG. 14B).

In an embodiment of the present disclosure, PD inhibits a broad range of primary isolates of HIV and SIV at submicromolar to micromolar concentrations with minimal cytotoxicity to human cells (CC₅₀/IC₅₀>300). In further embodiments, PD is effective against a broad range of primary HIV-1 isolates as well as HIV-2 (IC₅₀˜1 μM). In some embodiments, PD is fully active in cervical fluids. In an embodiment, PD exhibits low toxicity towards different human cells. In an embodiment, the cells are cervical cancer cells (CC₅₀>300 μM). In another embodiment of the present disclosure, PD is an effective in exhibiting it virucidal property against both cell-free and cell-associated virus. In a related embodiment, PD inhibits the transmission of dendritic cell-associated HIV-1 to T cells. In an embodiment of the present disclosure, PD retains antiviral potency in vitro prior to the addition of HIV-1 to the cells. In a related embodiment of the present disclosure, PD exhibits rapid antiviral action in vitro following infection of cells with HIV.

Additionally, the present disclosure pertains to PD as an anti-HIV microbicide. In an embodiment, PD is a stable and an effective microbicide at both acidic and neutral pH. In another embodiment, PD is fully active as a microbicide in the presence of seminal plasma and cervical fluids. In some embodiments, PD retains its full potency as a microbicide when stored in PBS under acidic pH at 42° C. for at least 8 weeks. In a further embodiment, PD can be formulated in hydroxyethyl cellulose (HEC) gel. In an embodiment of the present disclosure PD is non-toxic to the vaginal commensal bacteria Lactobacilli (CC₅₀>300 μM) and freshly activated PBMC (CC₅₀>200 μM). In an embodiment, PD is active in PBMCs against HIV-1 clinical isolates representing different viral subtypes and tropisms (average IC₅₀=0.55 μM). In all embodiments of the present disclosure, PD does not foster the emergence of resistant variants of HIV-1.

Accordingly, one aspect of the present disclosure that will be disclosed in more detail herein provides a method of inactivating virus particles in a biological sample. In some embodiments, the method comprises contacting the biological sample with an effective amount of an antiviral composition comprising PD 404,182. Notably, in an embodiment, the virus particle to be inactivated may be a retrovirus virus. Examples of retrovirus that may be inactivated by PD include but are not limited to HIV pseudotyped lentiviruses, primary human immunodeficiency virus-1 isolates (HIV-1), human immunodeficiency virus-2 (HIV-2), and simian immunodeficiency virus (SIV). In an embodiment of the present disclosure, the inactivation of the virus particle by PD 404,182 may be mediated by physical disruption of the virion. In some embodiments of the present disclosure, the virus particle may or may not be associated with a cell. In general, the inactivation of the virus particle by PD 404,182 occurs at a temperature range of about 25° C. to about 37° C. In some embodiments of the present disclosure, the biological sample requiring antiviral treatment may include blood, a blood product, cells, a tissue, an organ, sperm, a vaccine formulation, or a bodily fluid. In some embodiments, this method may be used to treat virus infected blood from a blood transfusion.

In another embodiment, the present disclosure provides a method of treating, preventing, or reducing a viral infection in a subject in need thereof. Such a method comprises administering to the subject a therapeutically or prophylactically effective amount of an antiviral composition comprising PD 404,182 or a pharmaceutically acceptable salt thereof. Specifically, this method is directed to treating, preventing, or reducing viral infections in humans. In various embodiments, the viral infections envisioned by the present disclosure include but are not limited to those caused by retroviruses. Specific examples of the retroviruses include but are not limited to HW pseudotyped lentiviruses, human immunodeficiency virus-1 (HIV-1), human immunodeficiency virus-2 (HIV-2), and simian immunodeficiency virus (SIV). In an embodiment of the present disclosure, the antiviral activity of PD 404,182 is independent of viral envelope proteins. In a related embodiment, the method disclosed herein may be effective against a co-infection with another virus. In an embodiment of the present disclosure, the method may be effective in treating, reducing or preventing co-infection by the Hepatitis C Virus (HCV) in a subject in need thereof.

In an additional embodiment, the method further comprises administering to the subject one or more additional therapeutic agents. In some embodiments of the present disclosure, the one or more additional therapeutic agents may be selected from the group consisting of interferon agent, ribavirin, HCV inhibitor, and HIV inhibitor. In some embodiments of the present disclosure the antiviral composition may be administered intravenously, orally, subcutaneously, intramuscularly or transdermally. In an embodiment, the antiviral composition may be administered transdermally. In an embodiment of the present disclosure, the antiviral composition is in the form of a gel, foam, cream, ointment, lotion, balm, wax, salve, solution, condom coated with the composition, suppository, suspension and spray.

Another aspect of the present disclosure relates to a method of preventing transmission of a viral infection to a subject in need thereof. In an embodiment of the present disclosure, the viral infection is caused by a retrovirus. In some embodiments, such a method comprises contacting a mucus membrane of the subject with a topical formulation comprising an effective amount of PD 404,182 or a pharmaceutically acceptable salt thereof. In some embodiments, the formulation disclosed herein may be in the form of a gel, foam, cream, ointment, lotion, balm, wax, salve, solution, suppository, formulation coated condom, suspension and spray. In certain embodiments, the mucus membrane is the mucus membrane of the cervix or of the rectum. In an aspect of the present disclosure, the method is effective in preventing transmission of viral infections caused by viruses selected from the group consisting of HIV pseudotyped lentiviruses, human immunodeficiency virus-1 (HIV-1), human immunodeficiency virus-2 (HIV-2), and simian immunodeficiency virus (SIV). In yet another embodiment, the method may also be effective against a viral infection caused by co-infection of the HIV and the HCV viruses. In all embodiments the method of the present disclosure exhibits low cytotoxicity.

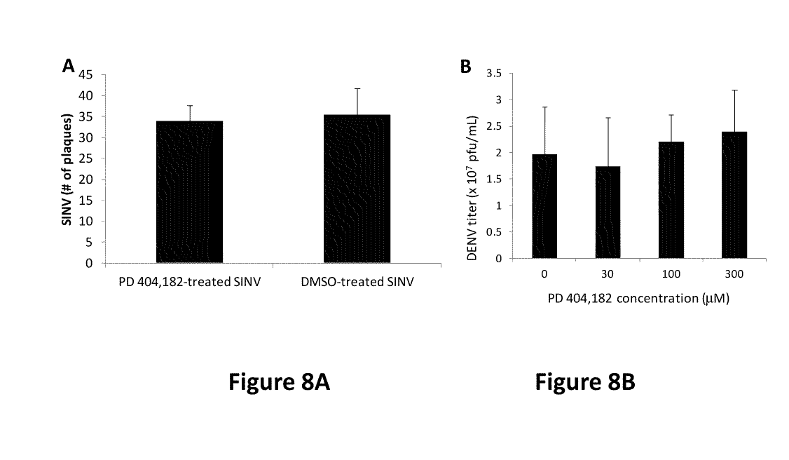

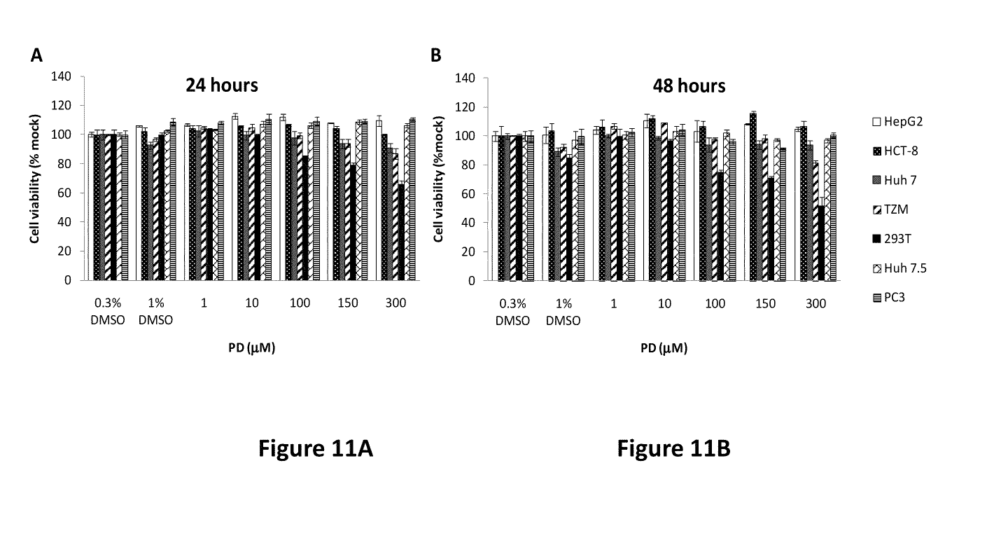

Hence, the present disclosure provides a small molecule, PD 404,182 (PD), which renders extracellular cell culture-produced HIV pseudotyped lentiviruses and several primary isolates of HIV and SIV non-infectious. In the case of HIV pseudotyped lentivirus and primary HIV-1, the antiviral activity of PD appears to be due to physical disruption of the virion. The antiviral action of PD is very rapid, as >99.5% of the lentivirus becomes inactivated within 5 min of contact with 300 μM PD at 37° C. However, only ˜40% of the lentiviruses are lysed in the same period, indicating that PD inactivates HIV pseudotyped lentiviruses and HIV-1 by physical disruption that does not necessitate complete lysis of virions. PD exposure does not appear to significantly rupture HCV or inhibit its attachment to cells, even with 90 min of exposure at 37° C. (FIGS. 7A-7B), despite inactivation of extracellular virus, suggesting a subtle disruption of virions (e.g. by irreversibly interfering with membrane fluidity or curvature) that causes inhibition of a post attachment step such as endocytosis or fusion with the endosomal membrane. Encouragingly, PD exhibits very low cytotoxicity in several human cell lines (CC50>300 μM; FIG. 11). The selectivity index (CC50/IC50) of PD is >300 for HIV and >27 for HCV. Intriguingly, despite exhibiting strong lysis of virions derived from the HIV capsid, PD does not directly lyse liposomes and shows no attenuation in antiviral activity when pre-incubated with liposomes, suggestive of little to no direct interaction with lipid membranes. The antiviral action of PD thus appears to be different from that reported for the amphipathic virucidal peptide CSA, which lyses both virions and liposomal membranes, and the membrane-intercalating virucidal molecule LJ001, whose antiviral effect is attenuated by pre-incubation with liposomes.

It is conceivable that PD disrupts the structural integrity of virions by selectively interacting with a feature of virions that involves interplay between two or more structural components (e.g. lipid membrane and envelope protein/capsid). There exists a possibility that PD may interfere with other virion structural components not represented in the liposome model, for example, sites that are glycosylated or phosphorylated.

The fact that the virion lysis activity of PD is temperature-dependent, suggests that a minimal level of viral membrane fluidity may be required to sufficiently compromise virion integrity to the point of viral RNA release. PD is inactivated by human serum and medium conditioned by human cell culture, possibly by interacting with one or more small molecules/peptides secreted by humans but not bovine cells. Since the inventors observed that PD retains its full antiviral activity in cervical fluids, the PD neutralizing molecule(s) present in human serum and conditioned cell culture growth medium is likely physiologically irrelevant in the case of development of PD as a topical microbicide for the prevention of HIV transmission.

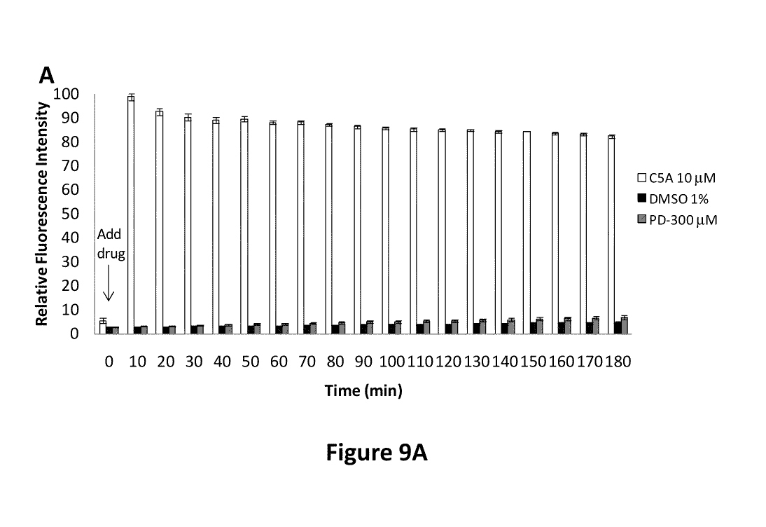

A striking feature of PD is its highly specific inactivation of certain viruses—HIV and related retroviruses and HCV were found to be inactivated—without strong association directly with or disruption of lipid membranes in general, as evident from our liposome studies. PD exhibited no significant antiviral effect on Dengue virus, an enveloped flavivirus closely related to HCV, or cell culture-produced Sindbis virus, an enveloped alphavirus (FIG. 8A-8B). Like HCV, Dengue virus acquires the viral envelope by budding into the ER lumen and is able to undergo intensive structural rearrangement in an infected cell. On the other hand, Sindbis virus, like HIV, buds from the plasma membrane and contains an envelope rich in cholesterol and sphingolipid molecules. The absence of non-specific cleavage of/association with lipid membranes may, at least in part, account for the molecule's very low cytotoxicity.

### Applications and Advantages

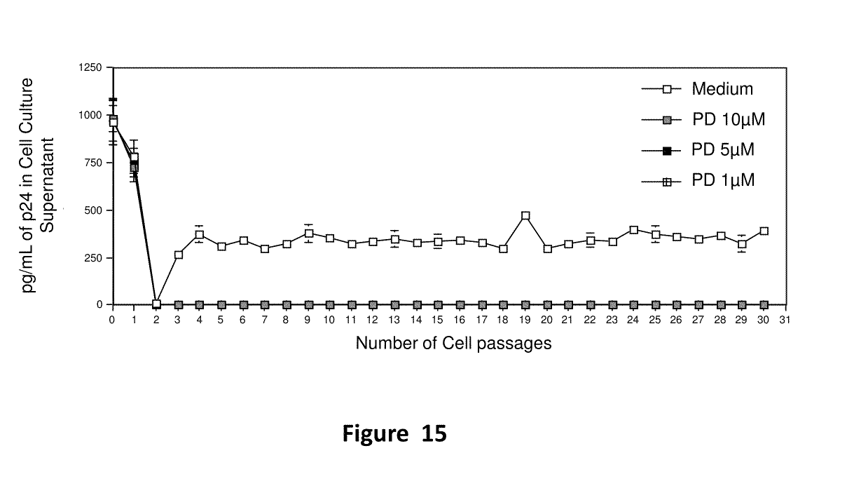

It is estimated that there are approximately four million new incidences of HIV infection each year, mostly transmitted through heterosexual intercourse. The development of a vaginal (or rectal) microbicide against HIV would represent a major stride towards slowing the global spread of HIV. The compound of the present disclosure possesses several desirable attributes that make it an attractive candidate anti-HIV microbicide. Most notably, PD 1) exhibits a unique mode of action—irreversible disruption of HIV through interaction with a yet unknown structural component; 2) exhibits antiviral activity against a broad range of primary HIV-isolates, HIV-2, and SIV at submicromolar to micromolar concentrations (Table 1); 3) retains its antiviral activity for at least 8 hours in cell culture at 37° C. prior to the addition of HIV-1 to the cells; 4) is effective against both cell-free and cell-associated HIV-1 and inhibits the transmission of dendritic cell-associated HIV-1 to T cells; 5) is potent at neutral and low pH and fully active in seminal plasma and cervical fluids; 6) is extremely efficacious since less than 5 min of incubation with virus results in >99% loss of viral infectivity; 7) is non-toxic to human cells; 8) is non-toxic to the commensal bacteria Lactobacilli (Table 2); 9) is specific—being ineffective against other enveloped viruses including Sindbis and Dengue virus, setting PD apart from non-specific surfactant/polyanion-based anti-HIV microbicides; 10) is viral-envelope-protein-independent; and 11) does not foster the emergence of resistant HIV-1 variants at sub-neutralizing concentrations in cell culture for 60 days (FIG. 15).

Drugs that target viral proteins mediating replication of viral nucleic acids or virus attachment to target cells often foster the emergence of escape mutants. The antiviral action of PD on critical components of the virus other than specific virus envelope proteins makes the development of drug resistant mutant viruses less likely. Several candidate anti-HIV microbicides exist, but only a handful exhibit an ability to strongly and irreversibly disrupt virions without being detrimental to cells. PD 404,182 is therefore an anti-HIV compound with a unique mode of action and represents a useful molecular scaffold for the generation of new anti-HIV-1 microbicides. Finally, the observation that PD is able to inactivate both HCV and HIV, and the unique antiviral action of this small molecule justifies further studies of PD and derivatives thereof to determine antiviral effects upon other enveloped and non-enveloped viruses.

Pharmaceutical compositions described herein can be used to provide effective topical antiviral activity. In certain embodiments, the compositions of the invention may reduce viral load at the infection site.

In other certain embodiment, the compositions may be in the form of gels, foams, creams, ointments, lotions, balms, waxes, salves, solutions, suspensions, sprays. The compositions may include other therapeutic agents. Thus, for example, the compositions may contain additional compatible pharmaceutically active agent for combination therapy (such as anti-viral, anti-microbial, anti-parasitic agents, anti-pruritics, astringents, healing promoting agents, steroids, anti-inflammatory agents) or may contain materials useful in formulating various dosage forms of the present invention, such as excipients, dyes, pigments, perfumes, fragrances, lubricants, thickening agents, stabilizers, skin penetration enhancers, preservatives, film forming polymers, or antioxidants. In general, the gel, cream, or ointment compositions may be, but not limited to, the following: a hydrophobic or hydrophilic ointment, and oil-in-water or a water-in-oil emulsion; thickened aqueous gels, hydrophilic gels

The preferred composition depends on the method of administration, and typically comprises one or more conventional pharmaceutically acceptable carriers, adjuvants, and/or vehicles (together referred to as “excipients”). Formulation of drugs is generally discussed in, for example, Hoover, J., Remington's Pharmaceutical Sciences (Mack Publishing Co., 1975) and Ansel's Pharmaceutical Dosage Forms and Drug Delivery Systems (Lippincott Williams & Wilkins, 2005).

Solid dosage forms for oral administration include, for example, capsules, tablets, pills, powders, and granules. In such solid dosage forms, the compounds or salts are ordinarily combined with one or more excipients. If administered orally, the compounds or salts can be mixed with, for example, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in, for example, a dispersion of the compound or salt in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings.

Liquid dosage forms for oral administration include, for example, pharmaceutically acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also can comprise, for example, wetting, emulsifying, suspending, flavoring (e.g., sweetening), and/or perfuming agents.

Parenteral administration includes subcutaneous injections, intravenous injections, intramuscular injections, intrasternal injections, and infusion. Injectable preparations (e.g., sterile injectable aqueous or oleaginous suspensions) can be formulated according to the known art using suitable dispersing, wetting agents, and/or suspending agents. Acceptable vehicles and solvents include, for example, water, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), and/or polyethylene glycols.

It will be understood by those skilled in the art that the compounds of the invention may be administered in the form of a composition or formulation comprising pharmaceutically acceptable carriers and/or excipients.

Formulations for parenteral administration may, for example, be prepared from sterile powders or granules having one or more of the excipients mentioned for use in the formulations for oral administration. A compound or salt of the invention can be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

Suppositories for rectal administration can be prepared by, for example, mixing a compound or salt of the invention with a suitable nonirritating excipient that is solid at ordinary temperatures, but liquid at the rectal temperature, and will therefore melt in the rectum to release the drug. Suitable excipients include, for example, cocoa butter; synthetic mono-, di-, or triglycerides, fatty acids, and/or polyethylene glycols.

Topical administration includes the use of transdermal administration, such as transdermal patches or iontophoresis devices.

Other excipients and modes of administration known in the pharmaceutical art also may be used.

Routes of administration include, but are not limited to, intravenous (iv), intraperitoneal, subcutaneous, intracranial, intradermal, intramuscular, intraocular, intrathecal, intracerebral, intranasal, transmucosal, or by infusion orally, rectally, via iv drip, patch and implant. Intravenous routes are particularly preferred.

The present invention also extends to forms suitable for topical application such as a gel, foam, cream, ointment, lotion, balm, wax, salve, solution, condom coated with the composition, suppository, suspension and spray.

The subject of the viral inhibition is a mammal, such as, but not limited to, a human, a primate, a livestock animal, for example, a sheep, a cow, a horse, a donkey or a pig; a companion animal for example a dog or a cat; a laboratory test animal, for example, a mouse, a rabbit, a rat, a guinea pig or a hamster; or a captive wild animal, for example, a fox or a deer. Preferably, the subject is a primate. Most preferably, the subject is a human.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved and (b) the limitations inherent in the art of compounding.

Procedures for the preparation of dosage unit forms and topical preparations are readily available to those skilled in the art from texts such as Pharmaceutical Handbook. A Martindale Companion Volume Ed. Ainley Wade Nineteenth Edition The Pharmaceutical Press London, CRC Handbook of Chemistry and Physics Ed. Robert C. Weast Ph D. CRC Press Inc.; Goodman and Gilman's; The Pharmacological basis of Therapeutics. Ninth Ed. McGraw Hill; Remington; and “The Science and Practice of Pharmacy”. Nineteenth Ed. Ed. Alfonso R. Gennaro Mack Publishing Co. Easton Pa.

### Additional Embodiments

Reference will now be made to more specific embodiments of the present disclosure and experimental results that provide support for such embodiments. However, Applicants note that the disclosure below is for illustrative purposes and is not intended to limit the scope of the claimed subject matter in any way.

### Example 1

### Reagents

PD 404,182 and Triton X-100 were purchased from Sigma-Aldrich (St. Louis, Mo.). PD 404, 182 is identified by CAS Number 72596-74-8, PubChem Substance ID 24278629, and has the empirical formula C₁₁H₁₁N₃S and has the following chemical structure:

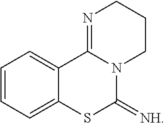 [SMILES: N=C1Sc2ccccc2C2=NCCCN12]

PD was dissolved in DMSO to a final concentration of 30 mM-40 mM, aliquoted and stored at −20° C. C5A was synthesized at the Scripps Research Institute. C5A was dissolved in 100% DMSO to final concentrations of 10 mg/mL, respectively, and stored at −20° C. Dulbecco's Phosphate-Buffered Saline (DPBS) and Penicillin-Streptomycin (pen-strep) were purchased from Thermo Scientific HyClone (Logan, Utah) and Lonza (Walkersville. MD), respectively. Unless otherwise stated, the complete growth media for all cell culture work was DMEM containing 4500 mg/L glucose, 4.0 mM L-Glutamine, and 110 mg/L sodium pyruvate (Thermo Scientific HyClone, Logan, Utah) supplemented with 10% fetal bovine serum (Atlanta Biologicals, Lawrenceville, Ga.) and 1× non-essential amino acids (Thermo Scientific HyClone, Logan, Utah).

293T cells were from Life Technologies (Grand Island, N.Y.). Vero cells were obtained from ATCC (Manassas, Va.). The following reagents were obtained through the NIH AIDS Reagent Program, Division of AIDS, NIAID, NIH: TZM-b1 from Dr. John C. Kappes, Dr. Xiaoyun Wu and Tranzyme Inc.; HIV-1 isolates 92RW016, 92RW21, 92TH006, 92TH026, 93TH053, 93BR020, 93BR021, 93BR029, 98IN017, 981N022, 92UG001, 92UG005, 92UG024, 92UG029, from The UNAIDS Network for HIV Isolation and Characterization; HIV-1 92HT599 from Dr. Neal Halsey; HIV-1 96USNG31 from Drs. D. Ellenberger, P. Sullivan, and R. B. Lal; HIV-1 RU132 from Dr. A. Bobkov and Dr. Jonathon Weber; HIV-1 931N101 from Dr. Robert Bollinger; and HIV-1Jv1083 from Dr. Alash'le Abimiku. All primary HIV isolates were amplified in activated human PBMCs. NL4.3 HIV was obtained from NIH AIDS Research and Reference Program.

### Example 2

### Production of HCVcc and Pseudotyped Lentiviruses

Production and titering of Jc1 HCVcc was as previously described. Unless otherwise specified, all lentiviral pseudoparticles were generated from 293T cells by co-transfection of plasmids carrying HIV gag-pol, a provirus (pTRIP-Gluc, pV1-Gluc or pV1-B), and an appropriate envelope protein. For example, pseudotyped lentiviruses were produced by co-transfecting 293T cells with plasmids carrying HIV gag-pol, a provirus (pV1-B1) or pTRIP-Gluc, and vesicular stomatitis virus glycoprotein (VSV-G). TransIT reagent (Mirus, Madison, Wis.) was used to perform the transfection following the manufacturer's protocol. The supernatants containing the pseudoparticles were collected 48 h post transfection, filtered (0.45 μm pore size) and stored at −80° C. until use.

For production of MLVpp, SINVpp, and HIVpp, plasmids encoding the viral envelope proteins pHIT456, pintron-SINV-env, and HIV BaL.01 were used, respectively. pV1 is a minimal HIV-1 provirus lacking most HIV genes except for all necessary cis acting sequences such as Tat, Rev and Vpu ORF. In pV1-B and pV1-Gluc, the Nef gene was replaced by an irrelevant peptide and the Gluc gene, respectively. The titer of VSV-Gpp and HIVpp harboring pV1-B or pV1-Gluc was measured on a TZM-b1 indicator cell line using the lacZ reporter in a limiting dilution assay.

### Example 3

### PD Stability

PD was diluted in buffered DPBS (pH 4, 6, 8, 10) or cervical fluids (pool of 3 donors, 5-fold diluted in DPBS) to achieve a final concentration of 30 μM. DPBS was buffered to the desired pH using hydrochloric acid or sodium hydroxide. Cervical fluids were collected and processed as previously described. Diluted drug was incubated at the desired temperature for 0, 24 or 48 h. After the temperature incubation, the drug mixture was further diluted to 1, 0.1 and 0.05 μM in complete growth media and used to incubate with VSV-G lentiviral pseudo particles (VSV-Gpp; viral supernatant diluted 500-fold in complete growth medium) at 37° C. for 30 minutes. Huh-7.5 (2×104 cells/well) seeded 24 h earlier were inoculated with the PD-treated virus at 4° C. for 2 h, thoroughly washed to remove unbound viruses and drug, replenished with complete growth media containing 1× pen-strep and returned to 37° C. and 5% CO2. Viral infectivity was quantified 48 hours later by measuring supernatant Gluc levels using the BioLux Gaussia Luciferase Assay Kit (New England Biolabs, Ipswich, Mass.).

To study the long-term stability of PD, the compound was diluted to 5 μM in DPBS buffered at pH 4 and 7 using acetic acid (0.1%) and HEPES (2.5 mM), respectively, aliquoted and incubated at 4° C., room temperature (RT) or 42° C. Each week an aliquot was removed and tested for antiviral activity as previously described above. Similar experiments were conducted with PD (5 μM) or vehicle control (0.02% DMSO) diluted in DPBS (adjusted to pH 4) containing 1.5% HEC.

### Example 4

### PD Stability in Seminal Plasma

TZM-b1 cells (10⁵ cells/well) were seeded in a flat-bottom 96-well plate. The next day, PD dilutions were prepared at a 2× concentration in seminal plasma (pool of 10 donors, 2-fold diluted in DMEM) and 100 μL of the 2×-concentrated mixtures were added to wells. Fifty microliters of a predetermined dilution of HIV stock (X4 NL4.3, 1 ng of p24) was placed in each test well. The cultures were incubated at 37° C. and 5% CO₂ for 4 hours, washed with complete growth medium to remove unbound viruses and compound, replaced with fresh growth medium, and returned to the incubator. Infection was scored 48 h later by β-galactosidase activity.

### Example 5

### Anti-HIV Efficacy Evaluation in Fresh Human PBMC

Testing of PD against HIV-1 in PBMCs was performed at Southern Research Institute as described previously. Briefly, PHA-stimulated cells from at least two normal donors were mixed together, diluted in fresh medium to a final concentration of 1×10⁶ cells/mL, and plated in a 96 well round bottom microplate at 50 μL/well (5×10⁴ cells/well). Test drug dilutions were prepared at a 2× concentration in microtiter tubes and 100 μL of the 2×-concentrated mixtures were added to wells. Fifty microliters of a predetermined dilution of virus stock was placed in each test well (final MOI˜0.1). Separate plates were prepared identically without virus for drug cytotoxicity studies. The PBMC cultures were maintained for seven days following infection at 37° C., 5% CO₂. After this period, cell-free supernatant samples were collected for analysis of reverse transcriptase activity, and compound cytotoxicity was measured by addition of 3-(4,5-dimethylthiazol-2-yl)-5-(3-carboxymethoxyphenyl)-2-(4-sulfophenyl)-2H-tetrazolium (MTS; CellTiter 96 Reagent, Promega) to the separate cytotoxicity plates for determination of cell viability. Wells were also examined microscopically and any abnormalities were noted.

### Example 6

### Lactobacillus Toxicity Testing

Reference strains of L. crispatus and L. jensenii were obtained from the ATCC (Manassas, Va.) and cultured on Columbia blood agar plates at 35° C. in air enriched with 6% CO₂. Bacterial suspensions were prepared in saline or ACES buffer [34] to a density of 2 McFarland units (˜2×10⁸ bacteria/mL), and exposed to PD (300 μM) or DMSO (10%) for 30 minutes at 37° C. After incubation, the cells were serially diluted in ACES buffer, pH 7.0 (Sigma-Aldrich, St. Louis, Mo.) and plated on blood agar plates to quantify colony forming unit per mL (CFU/mL)

### Example 7

### Primary Cells Toxicity Testing

Primary cells were seeded at 6×10³ cells/well in flat bottom 96-well plates in triplicates in the presence of increasing concentrations of PD. We used the permeabilization agent saponin (0.1%) as positive control. After 0, 7 and 14 days, the amounts of lactate dehydrogenase (LDH) in the cell culture media were quantified using the LDH Cytotoxicity Assay Kit (Cayman Chemical, Ann Arbor, Mich.).

Cell death can occur either by apoptosis or by necrosis. Necrosis is accompanied by mitochondrial swelling and increased plasma membrane permeability, whereas apoptosis involves an articulated breakdown of the cell into membrane-bound apoptotic bodies. Lactate dehydrogenase (LDH) is a soluble cytosolic enzyme that is released into the culture medium following loss of membrane integrity resulting from either apoptosis or necrosis. LDH activity, therefore, can be used as an indicator of cell membrane integrity and serves as a general means to assess cytotoxicity resulting from chemical compounds or environmental toxic factors. Cayman's LDH Cytotoxicity Assay Kit measures LDH activity present in the culture medium using a coupled two-step reaction. In the first step, LDH catalyzes the reduction of NAD to NADH and H⁺ by oxidation of lactate to pyruvate. In the second step of the reaction, diaphorase uses the newly-formed NADH and H⁺ to catalyze the reduction of a tetrazolium salt (INT) to highly-colored formazan which absorbs strongly at 490-520 nm.

### Example 8

### HCVcc Infection Assay

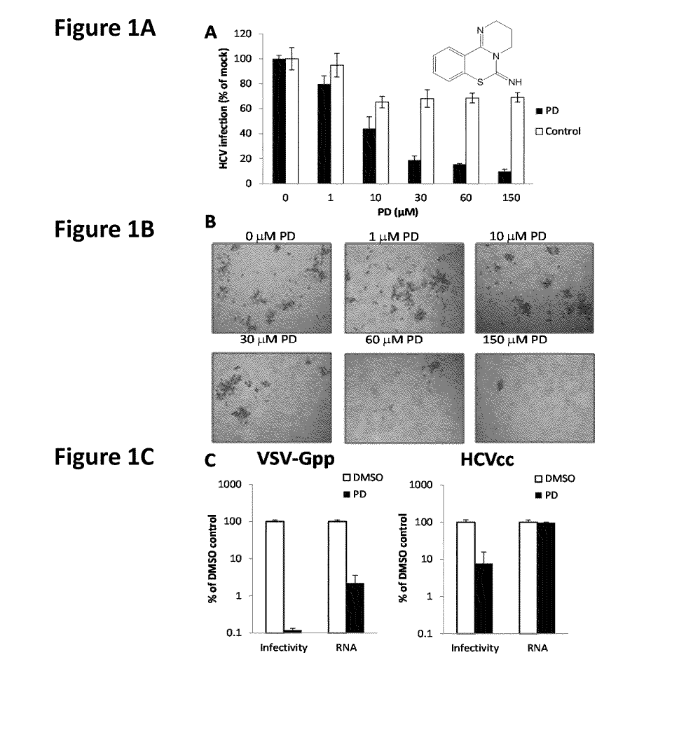

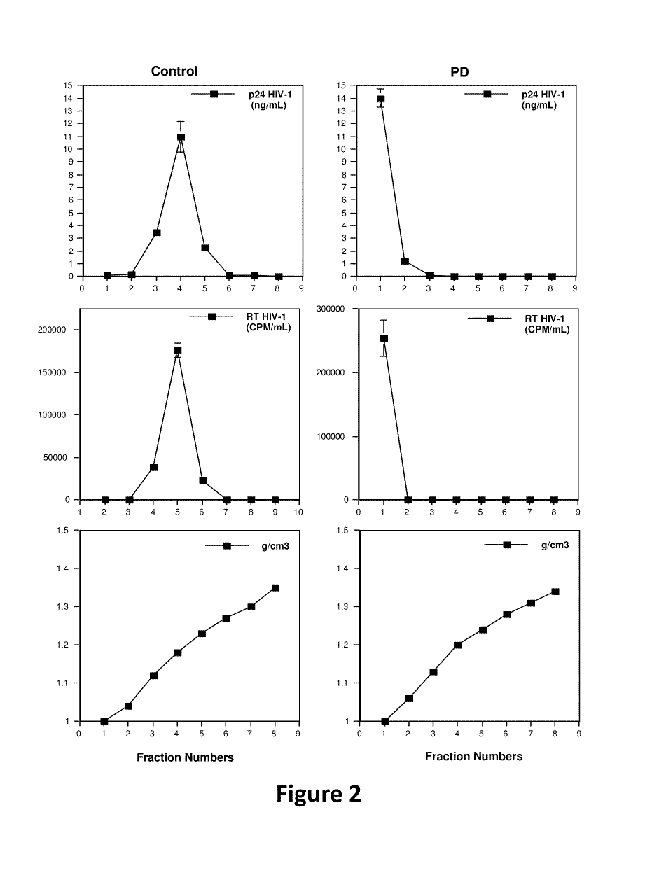

In FIG. 1A, Jc1 Gluc HCVcc (˜10⁵ TCID₅₀/mL) was concentrated 4-fold using a 100 kDa cut-off membrane ultracentrifugation column and washed twice with phenol red-free DMEM to remove any PD-inactivating molecules present in the virus supernatant. Concentrated virus was incubated with PD or DMSO at 37° C. for 30 minutes, diluted 1000-fold with fresh complete growth medium and used to infect naïve Huh-7.5 cells in 24-well (10⁵ cells/well) or 96-wp (2.8×10⁴ cells/well) plates 4-6 hours post seeding. The control samples contain virus and PD of the same final titers/concentrations, but with the virus and PD separately diluted 1000-fold prior to mixing. Viral infectivity was quantified by measuring the supernatant activity of the Gluc reporter or immunostaining infected cells for NSSA with 9E10 (anti-NSSA) antibody 72 h post infection.

### Example 9

### Spinoculation

PD-treated virus samples (HCVcc or VSV-Gpp) were cooled on ice for 5-10 min, and added to chilled target cells seeded in 96-well plates. Spinoculation was carried out at 300 g for 2 h at 4° C. After centrifugation, cells were washed 4 times with cold complete growth medium to remove any residual compound/unbound virus and returned to 37° C./5% CO₂.

### Example 10

### Viral RNA Quantification

For direct quantification of HCVcc/VSV-Gpp RNA, the total RNA from PD treated HCVcc/VSV-Gpp and cells infected with these viruses was isolated using the EZNA Viral RNA kit (Omega Bio-Tek) and Total RNA kit (Omega Bio-Tek), respectively. The amount of HCV RNA was quantified via TaqMan qRT-PCR (qScript One-Step FAST Kit, Quanta Biosciences, Gaithersburg, Md.) using previously described primers. The amount of lentiviral RNA was quantified using SYBR Green qRT-PCR (One-Step SYBR Green Kit, Quanta Biosciences) with primers pV1-qPCR-F, 5′-A C G G C C T C T A G A A T G A G C-3 and pV1-qPCR-R, 5′-A C A G C T G C T C G A G G T T-3.

Due to the large amount of residual provirus-encoding DNA present in the pseudoparticle preps obtained from transfected 293T cells, inventors were not able to directly quantify the viral RNA. Instead, repackaged pseudoparticles were used in all experiments involving direct quantification of viral RNA by qRT-PCR. Briefly, VSV-Gpp constructed from pV1-B was used to transduce Huh-7.5 cells. Three days later, these Huh-7.5 cells were transfected with plasmids carrying HIV gag-pol and VSV-G envelope protein to produce freshly repackaged pseudoparticles. Pseudoparticles serially repackaged in this manner at least 3 times were used in experiments requiring direct quantification of viral RNA.

### Example 11

Liposome Dye Release Assay.

Liposomes composed of 36 mg POPC, 39 mg DPPC, 4 mg POPS and 21 mg cholesterol per 100 mg, without or with 100 mM SulfoB (Avanti Polar Lipids, Inc), were prepared as described previously and sized via repeated extrusion through a 100 nm polycarbonate membrane filter (Avanti Polar Lipids, Inc). Dye release assays were performed in a Gemini EM Spectrofluorometer (Molecular Devices, San Francisco Calif.). 1 μL PD (30 mM), 0.24 μL C5A (10 mg/mL) or 1 μL DMSO were added to 100 μL liposomes (100 μM, 0.06 mg/mL) in PBS in 384-well plates and membrane disruption was gauged from the increase in SulfoB fluorescence at excitation/emission wavelength settings of 544/590 nm 5 minutes post-treatment. The fluorescence intensity corresponding to 100% SulfoB release was obtained by liposome disruption with 0.1% Triton X-100.

### Example 12

### HIV-1, HIV-2, SIV Infectivity Assays

TZM-b1 cells (100,000 cells/mL) were exposed to HIV or SIV (1 ng of p24/p27) for 4 h together with increasing concentrations of PD or DMSO control, washed, and infection was measured 48 h later by β-galactosidase activity. Primary HIV-1, HIV-2, and SW were obtained through the National Institutes of Health (NIH) AIDS Research and Reference Reagent Program and amplified in activated human peripheral blood mononuclear cells (PBMC, activated by PHA/IL-2 treatment). To determine the anti-HIV effect of PD in genital fluids, the same TZM cells were exposed to JR-CSF (1 ng of p24) for 4 h together with increasing concentrations of PD diluted in cervical fluids or seminal plasma

### Example 13

### HIV-1 Sedimentation Assay

Purified HW-1 (20 ng of p24 of NL4.3) was microcentrifuged for 90 min at 4° C. to remove free capsid, resuspended in PBS, exposed to PD or DMSO medium control, and loaded over a 20-70% sucrose gradient. After ultracentrifugation at 20,000 rpm for 24 h in a SW-41 T rotor, fractions (1 mL) were collected and tested for their content of viral proteins. HIV-1 capsid was detected by p24 ELISA. Reverse transcriptase (RT) was detected by exoRT assay. The density of each sucrose gradient fraction was determined by measuring the refractive index.

### Example 14

### HIV-1 Cell-to-Cell Transfer Assay

Blood-derived immature DC were plated at 50,000 cells per well in 96-well V-bottom plates (BD Biosciences). Cells were incubated with wild-type NL4.3-eGFP (X4), NL4.3-BaL-eGFP(R5), or the single-round NL4.3AEnv-eGFP pseudotyped virus with NL4.3 gp160 (X4) (25 ng of p24) for 2 h at 37° C. Medium supplemented with either PD or DMSO was then added and incubated with DC for 2 h. Cells were washed three times with warm medium, and CCR5_Jurkat T cells (100,000 cells) were added. Cells were cultured in a flat-bottom 96-well plate, harvested after 3 days, and fixed in 4% paraformaldehyde/PBS, and GFP expression was measured by FACS. Percentage of infected Jurkat T cells was selectively quantified by gating T cells using an anti-CD3 antibody. Virus isolates used in this study were pNL4.3-BaL (R5) in which wild-type NL4.3 envelope was switched for the R5BaL envelope, the pNL4.3 Env, which lacks gp160, the pNL4.3-eGFP (X4) and the pNL4.3-BaL-eGFP (R5), which encode the GFP gene instead of the Nef gene.

### Example 15

### Drug Resistance Study

HIV-1 (NL4.3, 1 ng of p24, corresponding to approximately 1,000-5,000 infectious units) was added to TZM-b1 cells (1×10⁶ cells). Fifteen minutes later, an aliquot of supernatant (50 μL) was collected for viral input normalization, and PD was added to the cells at 1, 5, or 10 μM. TZM-b1 cells were split every two days for a period of 60 days. Fresh PD was added at each passage to maintain the same concentration throughout the 60 days. Before each passage an aliquot of supernatant (50 μL) was collected to determine amount of virus in cell culture via p24 ELISA (Perkin Elmer Life Sciences).

### Example 16

### Statistical Analysis

Statistical significance between different samples was evaluated using Student's t-test in Microsoft Excel. A P value of 0.05 was considered statistically significant.

### Example 17

### PD 404,182 is Virucidal Against HCV and Pseudotyped Lentiviruses

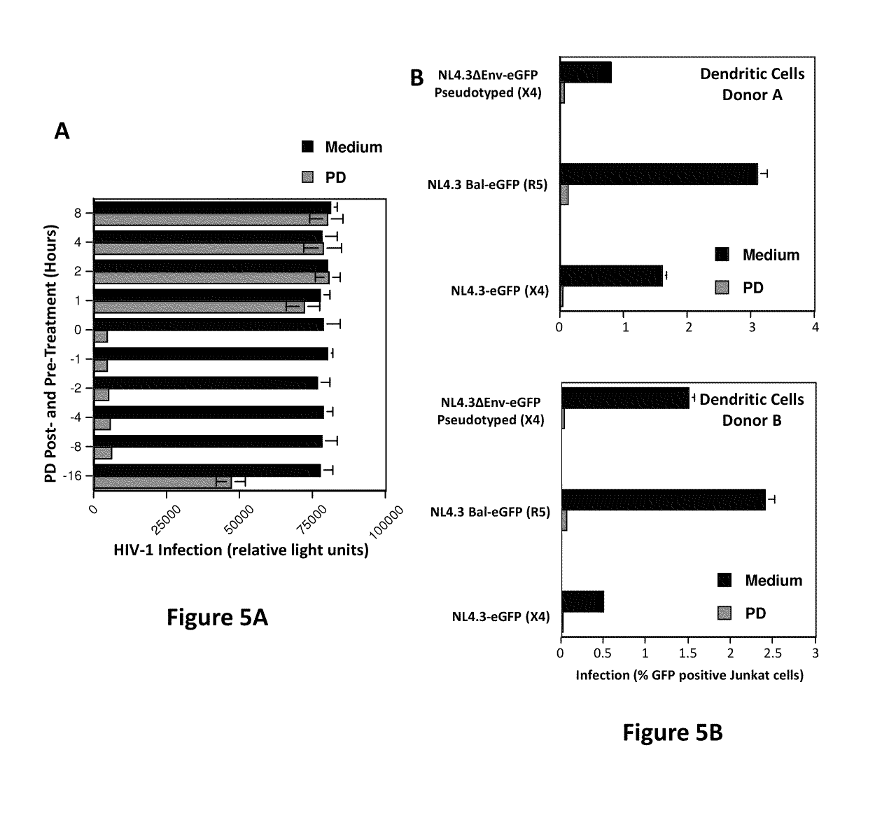

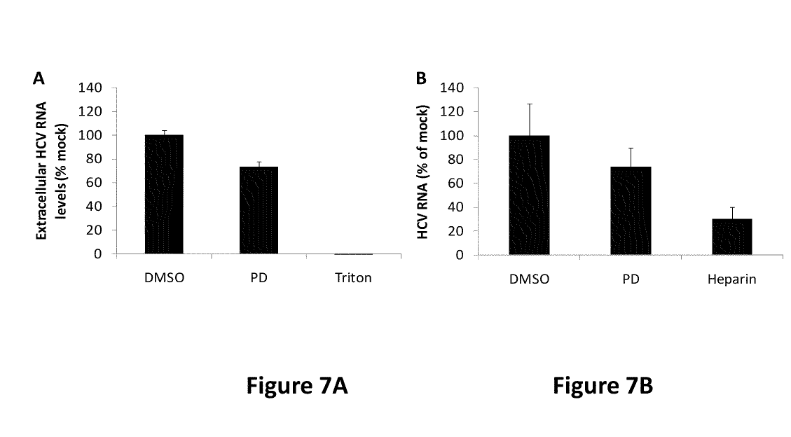

Previously, it has been shown that PD alleviates HCVcc-induced cytopathic effect and inhibits the cellular entry of HIV lentivirus pseudotyped with envelope glycoprotein from the H77 isolate of HCV and vesicular stomatitis virus (VSV). In this disclosure, the inventors show that PD inhibits HCV infection by inactivating extracellular virions. As shown in FIGS. 1A and 1B, PD dose-dependently inactivates cell-free HCVcc with an IC50 value of 11 μM. To explore the possibility that the antiviral activity of PD is independent of the viral envelope protein, PD was incubated with HIV lentiviruses pseudotyped with three additional envelope proteins derived from murine leukemia virus (MLV), Sindbis virus (SINV) and HIV. PD exhibits similar antiviral activity against all of these HIV pseudotyped lentiviruses (FIG. 6), indicating that the antiviral activity may derive from interference with a viral structural component other than the envelope proteins. Next, inventors evaluated whether PD treatment causes lysis of the virus membrane/capsid. Supernatant containing lentivirus pseudotyped with vesicular stomatitis virus envelope glycoprotein (VSV-Gpp) or HCVcc was treated with PD or with the corresponding concentration of the solvent DMSO at 37° C. for 30 min in the presence of RNase A prior to quantification of viral RNA and infectivity. As shown in FIG. 1C, treatment with 150 μM PD induces RNase-mediated degradation of VSV-Gpp RNA by ˜30-fold (3.7% remaining), and inhibits supernatant infectivity by ˜1000-fold (0.1% remaining), relative to DMSO-treated virus. The significant fold difference between virus inactivation and virion lysis, which becomes more pronounced with shorter virus-PD pre-incubation times (FIG. 4B), suggests that virion lysis is not required for PD-mediated virus inactivation. A similar effect was observed with the virucidal peptide CSA, which showed ˜100-fold more virion inactivation than lysis. Little to no virion lysis was observed in 150 μM PD-treated HCVcc, despite >10-fold inhibition of virus infectivity. A low but reproducible level of HCVcc virion lysis (27%) was observed (FIG. 7A) only when a higher concentration of PD (300 μM) was used in combination with a prolonged incubation at 37° C. (90 min). Since PD appears to significantly inactivate but only poorly lyse HCVcc, the inventors next determined whether PD-treated HCVcc particles that resist lysis by PD retain their ability to attach to the surface of cells. Measurement of cell surface-associated virus via qRT-PCR revealed that PD-treated HCVcc binds to cells comparably to control DMSO-treated virus, suggesting that treatment with the compound likely inhibits a post-attachment step for virions that remain intact (FIG. 7B). The ability of PD to physically inactivate both VSV-Gpp and HCVcc, two very different viruses, combined with the observation that the compound does not seem to significantly distinguish between HIV lentiviruses pseudotyped with different envelope proteins (FIG. 6), suggests that its antiviral activity is likely mediated through a common non envelope-protein structural component.

To evaluate the antiviral specificity of PD, inventors tested the effect of the compound on two other enveloped viruses—Sindbis virus (SINV), an alphavirus, and Dengue virus (DenV), a flavivirus closely related to HCV. As shown in FIG. 8A-8B, PD exhibits no significant inhibitory effect on the infectivity of SINV and DenV at 300 μM. Interestingly, despite the observed absence of antiviral activity against SINV, PD was found to exhibit strong antiviral activity against lentivirus pseudotyped with SINV envelope protein (FIG. 6), underscoring the non-specific nature of the antiviral effect on HIV pseudotyped lentiviruses. The neutrality of PD towards SINV and DenV suggests that PD may exert its antiviral effect by specifically interfering with a structural feature common to HCVcc and HIV pseudotyped lentiviruses but not present on SINV and DenV.

### Example 18

### PD Inactivates a Broad Range of Primary HIV Isolates and Related Retroviruses

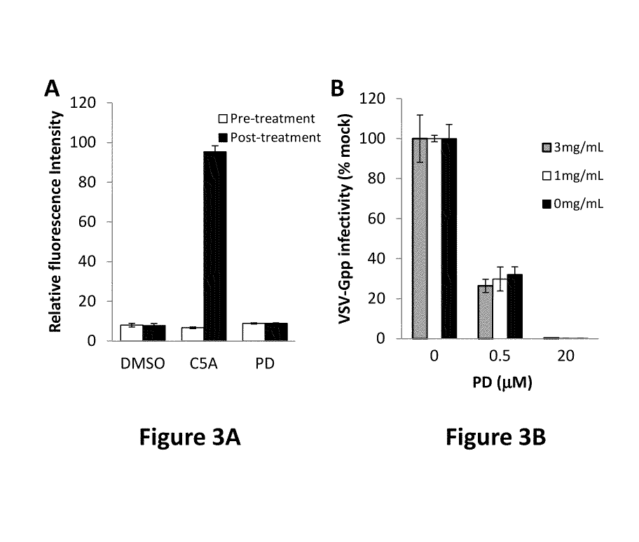

Since PD strongly inactivates all the HW pseudotyped lentiviruses regardless of the envelope protein, inventors tested (FIG. 6) and determined whether PD also inactivates primary HIV and related retroviruses. Using CD4+ HeLa cells (TZM-b1 cells (34)) that produce β-galactosidase in response to HIV infection, the antiviral activity of PD on 14 isolates of HIV-1 which represent various subtypes and which use different co-receptors—either CCR5 (R5 viruses) or CXCR4 (X4 viruses)—to infect cells, as well as isolates of other retroviruses including HIV-2 and simian immunodeficiency virus (SIV), was determined. Viruses were added to TZM-b1 cells together with PD for 4 h, cells were washed, and infection was scored 48 h later. As shown in Table 1, PD effectively inhibits all the tested isolates of HIV and SIV at submicromolar to low micromolar concentrations, on par with the potency of the virucidal amphipathic peptide CSA. Similar anti-HIV potency was observed when PD was diluted in cervical fluids (Table 1). To probe the effect of PD on the structural integrity of HIV particle, the inventors carried out a virus sedimentation assay. Purified HIV-1 (X4 NL4.3) (20 ng of p24 in PBS) was incubated in the presence or absence of PD (10 μM) for 30 min at 37° C. and loaded onto a 20-70% sucrose gradient. Each fraction was analyzed for the amount of HIV capsid and reverse transcriptase (RT) (FIG. 2). Untreated virus (capsid and RT proteins) sediments at a density of 1.16 g/cm³. In contrast, viral capsid and RT relocate to the top of the gradient in PD-treated virus preps, indicating that PD exerts its virucidal effect on HIV-1 and retroviral particles by compromising virion integrity. This observation is consistent with the lysis of HIV pseudotyped lentivirus shown in FIG. 1C.

### Example 19

### PD does not Lyse or Interact with Liposomal Membranes

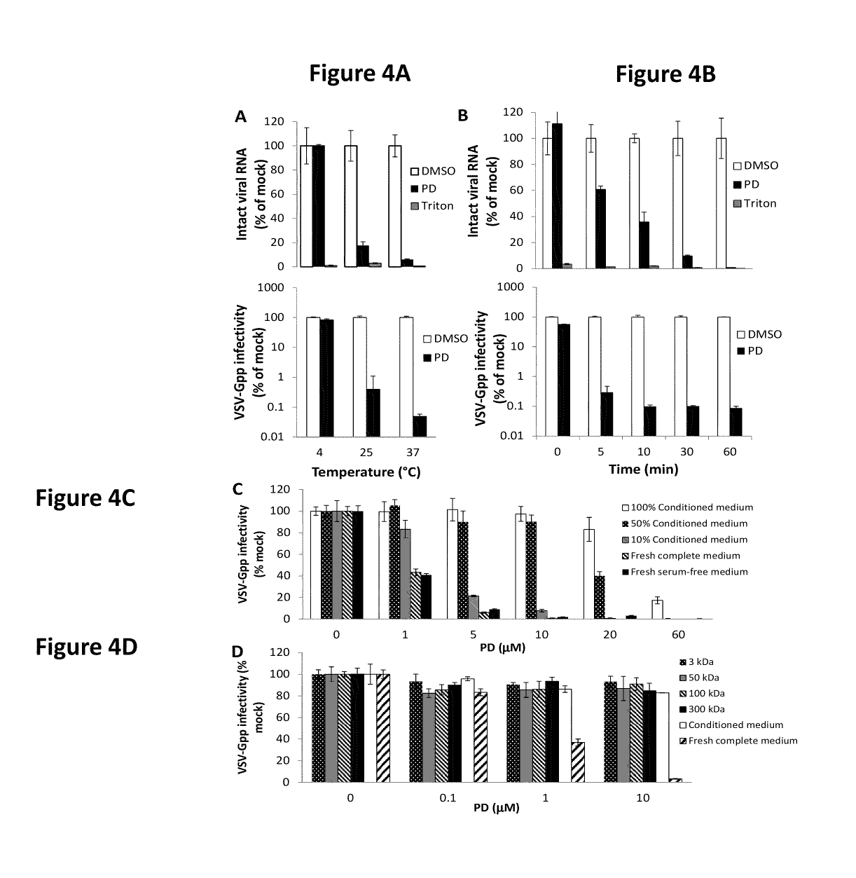

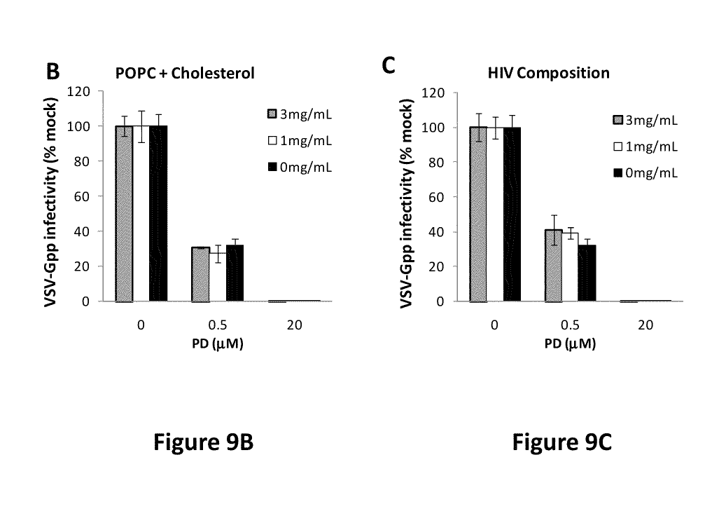

Because the anti-viral potency of PD is virus envelope protein independent, the inventors investigated the possibility that PD exerts its antiviral activity via disruption of the viral lipid membrane. Cholesterol-phospholipid liposomes entrapping the fluorescent dye sulforhodamine B (SulfoB) were incubated with PD, C5A or DMSO. Disruption of the liposomes is accompanied by dequenching of the fluorescent dye, and was quantified by measuring the resultant fluorescence release. C5A, a peptide derived from HCV NS5A protein, has previously been shown to lyse liposomes, and was used as a positive control. As shown in FIG. 3A, PD is unable to permeablize liposomes after incubation for 5 mins. No significant increase in intensity was observed even after prolonged (up to 3 h) incubation with PD (FIG. 9A).

Next, the Applicants set out to determine whether PD associates with liposomal membranes without causing lysis. Since PD is not inherently fluorescent, one cannot directly measure the interaction of PD with liposomes. Instead, inventors sought to determine whether the inhibitory effect of PD during infection can be reversed by the addition of liposomes. VSV-Gpp (3.4×10⁴ TCID₅₀/mL) mixed with PD and increasing concentrations of liposomes was used to infect Huh-7.5 cells. The presence of liposomes was not able to reverse PD's antiviral effect (FIG. 3B), suggesting that PD does not significantly interact with liposomes. In fact, liposomes of different lipid compositions were tested, but none were found to reverse the antiviral effect of PD (FIGS. 9B&9C).

### Example 20

### The Virucidal Activity of PD is Temperature-, Time- and Virus Dilution-Dependent

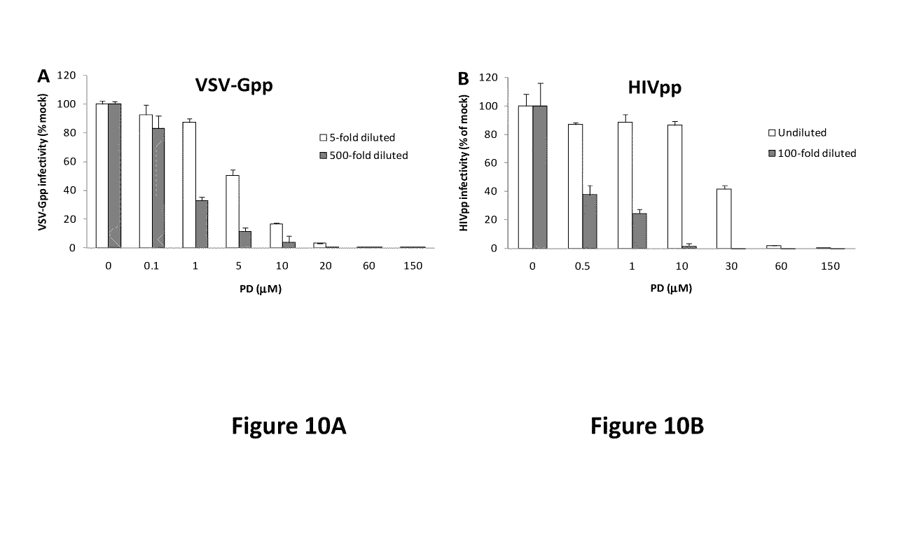

To further elucidate the antiviral effect of PD, VSV-Gpp was incubated with PD (300 μM) or DMSO (1%) at various temperatures and for various times, and the amount of remaining viral RNA and the infectivity of the virus/compound mixtures were determined thereafter. The virion lysis activity of PD was found to be temperature-dependent as PD disrupts the virus following a 30-min incubation at 37° C., but is less disruptive at 25° C. and exhibits no measurable virion lysis at 4° C. (FIG. 4A), indicating that a minimum level of membrane fluidity may be required for PD to lyse the virus membrane/capsid. Similar trend was observed for viral infectivity. PD rapidly inactivates HIV pseudotyped lentivirus as determined by the loss of viral RNA and infectivity (FIG. 4B). More than 99.5% of the VSV-Gpp was inactivated within 5 min when in contact with 300 μM PD. However, only ˜40% of the virions were compromised to the point of genomic RNA release for the same 5 min virus-PD pre-incubation, indicating that viron lysis is not required for virus inactivation. The sensitivity of virus to PD is also virus dilution-dependent (FIG. 10A-10B). The IC50 values of PD for cell culture-produced VSV-Gpp virus stocks diluted 5-fold and 500-fold in fresh complete growth medium (DMEM+10% FBS) are 4.6 μM and 0.5 μM, respectively. Similarly, the IC50 values for HIVpp (lentivirus pseudotyped with envelope protein from Ba1.01 HIV) are 24.6 μM and 0.3 μM for undiluted and 100-fold diluted virus. Further studies demonstrated that PD is inactivated by a molecule(s) present in conditioned cell culture medium, as virus diluted in conditioned medium is significantly less sensitive to inactivation by PD than the same virus diluted in fresh complete medium (FIG. 4C). To gauge the approximate size of the molecule(s) responsible for neutralizing the antiviral effect of PD, Applicants fractionated conditioned medium from Huh-7.5 cells by passage through ultrafiltration membranes with different pore sizes, and found that the filtrate from a 3 kDa membrane is able to inactivate PD to the same extent as the unfiltered conditioned medium (FIG. 4D). This result suggests that the molecule(s) responsible for neutralizing PD is relatively small (≦3 kDa). Although the presence of 10% fetal bovine serum appears to have no inhibitory effect on PD's antiviral activity (FIG. 4C), 10% human serum yielded a significant (50-80 fold) increase in IC50 (FIG. 10), suggesting the presence of PD-inhibitory factors in human serum in addition to cell culture media conditioned by human cancer cells.

### Example 21

### Efficacy of PD in Seminal Plasma

PD effectively inhibits several isolates of HIV-1 and SIV in TZM-b1 cells at submicromolar to low micromolar concentrations (IC₅₀˜1 μM) when diluted in DMEM or cervical fluid. It has been shown that seminal plasma can enhance HIV infectivity and protect HIV against the action of microbicides. Applicants therefore sought to test the antiviral activity of PD in seminal fluids. Briefly, CD4+CCR5+HeLa cells (TZM-b1 cells) that produce β-galactosidase in response to HIV infection were exposed to 14 different clinical and laboratory isolates of HIV-1, representing various subtypes that use either co-receptor CCR5 (R5 viruses) or CXCR4 (X4 viruses), in the presence of PD or DMSO prepared in 50% seminal plasma. After a 4 h incubation of the virus and compound with the cells, cells were washed and the infection was scored 48 h later by f3-galactosidase activity. The IC₅₀ and IC₉₀ of PD against the tested subtypes of HIV-1 range from 0.42-1.96 μM and 1.58-7.19 μM, respectively (Table 1). These values are consistent with those of PD's anti-HIV activity determined in DMEM (0.33-1.80 μM for IC₅₀ and 1.4-6.6 μM for IC₉₀) and in cervical fluid (0.61-2.30 μM for IC₅₀ and 1.80-7.50 μM for IC₉₀), indicating that seminal plasma does not negatively impact PD's anti-HIV activity.

It is worth noting that similar IC50 and IC90 values were obtained with PD diluted in DMEM or cervical fluids (Table 1), indicating that cervical fluids and seminal plasma are free of molecule(s) that detectably inhibit the antiviral activity of PD.

| Primary HIV-1 Isolate | | | PD, μM | | | | | |
|---|---|---|---|---|---|---|---|---|
| | | Coreceptor | DMEM¹ | | Cervical Fluid² | | Seminal Plasma³ | |
| Isolate | Clade | usage | IC₅₀ (μM) | IC₅₀ (μM) | IC₅₀ (μM) | IC₅₀ (μM) | IC₅₀ (μM) | IC₅₀ (μM) |
| 92RW021 | A | R5 | 0.43 +/− 0.03 | 3.8 +/− 0.3 | 0.67 +/− 0.04 | 4.4 +/− 0.2 | 0.58 +/− 0.04 | 4.62 +/− 0.32 |
| 92UG029 | A | X4 | 1.18 +/− 0.02 | 4.4 +/− 0.2 | 1.47 +/− 0.1 | 5.3 +/− 0.3 | 1.33 +/− 0.02 | 4.71 +/− 0.26 |
| 92TH026 | B | R5 | 0.35 +/− 0.01 | 2.8 +/− 0.2 | 0.61 +/− 0.03 | 3.3 +/− 0.1 | 0.43 +/− 0.02 | 2.95 +/− 0.16 |
| 92TH599 | B | X4 | 1.8 +/− 0.1 | 5.9 +/− 0.3 | 2.2 +/− 0.04 | 6.4 +/− 0.5 | 1.93 +/− 0.03 | 6.31 +/− 0.52 |
| 93IN101 | C | R5 | 1.5 +/− 0.2 | 5.1 +/− 0.3 | 1.8 +/− 0.1 | 5.5 +/− 0.2 | 1.76 +/− 0.02 | 5.36 +/− 0.37 |
| 93IN017 | C | X4 | 0.4 +/− 0.1 | 1.9 +/− 0.2 | 0.7 +/− 0.05 | 2.4 +/− 0.2 | 0.45 +/− 0.05 | 2.09 +/− 0.19 |
| 92UG005 | D | R5 | 1.26 +/− 0.2 | 5.3 +/− 0.4 | 1.39 +/− 0.11 | 5.7 +/− 0.3 | 1.32 +/− 0.02 | 5.57 +/− 0.44 |
| 92UG024 | D | X4 | 3.33 +/− 0.02 | 1.4 +/− 0.2 | 0.55 +/− 0.04 | 1.8 +/− 0.2 | 0.42 +/− 0.03 | 1.58 +/− 0.2 |
| 92TH006 | E | R5 | 1.8 +/− 0.2 | 6.6 +/− 0.4 | 2.3 +/− 0.1 | 7.6 +/− 0.6 | 1.96 +/− 0.01 | 6.73 +/− 0.51 |
| 93TH053 | E | X4 | 1.4 +/− 0.2 | 5.3 +/− 0.3 | 1.8 +/− 0.2 | 6.4 +/− 0.4 | 1.67 +/− 0.02 | 6.65 +/− 0.48 |
| 93BR029 | F | R5 | 0.7 +/− 0.1 | 3.5 +/− 0.2 | 1.2 +/− 0.1 | 4.6 +/− 0.3 | 0.85 +/− 0.01 | 3.72 +/− 0.26 |
| 93BR020 | F | X4 | 1.4 +/− 0.2 | 6.9 +/− 0.4 | 1.9 +/− 0.2 | 7.5 +/− 0.4 | 1.61 +/− 0.02 | 7.19 +/− 0.62 |
| RU132 | G | R5 | 0.7 +/− 0.2 | 3.1 +/− 0.2 | 0.9 +/− 0.2 | 3.9 +/− 0.5 | 0.74 +/− 0.01 | 3.28 +/− 0.22 |
| Jv1083 | G | R5 | 1.1 +/− 0.2 | 3.9 +/− 0.3 | 1.5 +/− 0.2 | 4.7 +/− 0.1 | 1.27 +/− 0.01 | 4.22 +/− 0.39 |

| | | |
|---|---|---|
| Other retroviruses | | |
| SIVmac251 32H | 1.1 +/− 0.2 | 6.2 +/− 0.4 |
| SIVsyk1.2 | 1.9 +/− 0.3 | 5.4 +/− 0.2 |
| HIV-20DC310342 | 1.8 +/− 0.2 | 4.7 +/− 0.2 |
| HIV-27312A | 2.2 +/− 0.3 | 6.8 +/− 0.3 |

TZM-bl cells (1 × 10⁵ cells/mL) were exposed to the indicated HIV isolates (1 ng of p24) in the presence of PD or DMSO diluted in 50% seminal plasma. Cells were washed 4 h post inoculation and fresh growth media was added. Infection was scored 48 h later by β-galactosidase activity.
Errors represent the SD of 2 independent experiments carried out in duplicate.
¹IC50 and IC90 are measured with PD diluted in DMEM with 10% fetal bovine serum.
²IC50 and IC90 are measured with PD diluted in cervical fluids (pool of 4 donors).
³IC₅₀ and IC₉₀ are measured with PD diluted in seminal plasma (pool of 4 donors).

### Example 22

### Antiviral Effect of PD Before, During, and After HIV-1 Exposure and on Cell-to-Cell Transmission of HIV-1

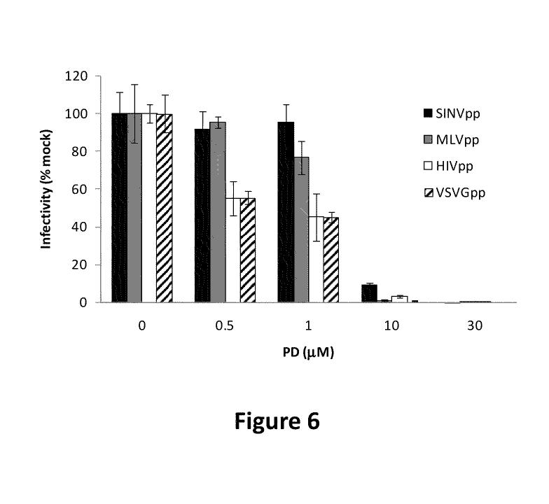

PD exhibits strong virucidal activity against HIV pseudotyped lentivirus and primary HIV, raising the possibility of its use as a topical microbicide for preventing the sexual transmission of HIV-1. To shed light on this possibility, the antiviral effect of PD when the compound was added to cells at various time points relative to the addition of HIV-1 was investigated. PD was added to TZM-b1 cells at 1, 2, 4 or 16 h before the addition of HIV-1 (R5 JR-CSF) (ing of p24), together with the virus (time zero), and at 1, 2, 4 or 8 h after the addition of the virus, and infectivity was quantified 48 h post virus addition. As shown in FIG. 5A, PD significantly inhibits HIV-1 infection when added together with the virus (time zero), and retains its full potency up to 8 h before the addition of the virus. However, PD loses its antiviral effect when added to cells after virus inoculation (FIG. 5A, 1, 2, 4 and 8 h post treatment). This result suggests that PD is not able to disrupt intracellular virus. In addition, extended (>16 h) preincubation of PD with cells prior to virus inoculation also significantly reduces the compound's antiviral efficacy. Since HIV-1 can be transmitted either as a cell-free or cell-associated virus, we examined the effect of PD on cell-to-cell transmission. Specifically, the capacity of PD to prevent dendritic cells (DC)-mediated transmission of HIV-1 was examined. For this purpose, a replication-defective virus (NL4.3AEnv-eGFP), that does not encode Env, but which has been pseudotyped with HIV-1 Env was used. (These pseudotyped viruses infect cells because they contain Env). Therefore, cells that have been infected by pseudotyped viruses cannot produce infectious viruses because de novo viruses do not encode Env. Thus, the use of pseudotyped viruses allowed the analysis of the effect of PD on the transmission of infectious particles from DC to T cells independently of DC infection. DC were incubated with wild-type NL4.3-eGFP (X4 virus) and NL4.3-BaL-eGFP (R5 virus) or pseudotyped NL4.3AEnv-eGFP/gp160 Env viruses (25 ng of p24). Two hours later, at which time the attachment of the virus onto DC is completed, PD (10 μM) was added. After 2 h, DC were washed to remove both free virus and PD. To measure DC-T cell transmission, Jurkat T cells were added for 3 days, and the percentage of infected T cells (gated with an anti-CD3 antibody) was analyzed by FACS. Only pseudotyped viruses that have been rapidly transferred from DC to T cells through the virological synapse (independently of DC infection) can infect T cells. Indeed, progeny viruses from DC infected by pseudotyped viruses can no longer infect T cells because they do not encode Env. Because DC were washed before adding T cells, the T cell infection by the pseudotyped virus observed in FIG. 5B could only arise from pseudotyped particles that were transferred from DC to T cells. Importantly, PD added to DC prevents subsequent T cell infection with the pseudotyped virus. This finding suggests that PD can also inactivate DC-bound virus, preventing HIV-1 transmission from DC to T cells. These results suggest that, unlike neutralizing antibodies, PD blocks cell-to-cell transfer of HIV-1 even when transmission occurs via the virological synapse.

### Example 23

### PD does not Lyse or Interact with Liposomal Membranes

Because the antiviral potency of PD is virus envelope protein-independent, the possibility that PD exerts its antiviral activity via disruption of the viral lipid membrane was investigated. Cholesterol-phospholipid liposomes entrapping the fluorescent dye sulforhodamine B (SulfoB) were incubated with PD, C5A or DMSO. Disruption of the liposomes is accompanied by dequenching of the fluorescent dye, and was quantified by measuring the resultant fluorescence release. PD does not interfere with Sulfo B fluorescence (data not shown). C5A, a peptide derived from HCV NS5A protein that has been shown to lyse liposomes, was used as a positive control. As shown in FIG. 3A, PD is unable to permeabilize liposomes after incubation for 5 mins. No significant increase in fluorescence intensity was observed even after prolonged (up to 3 h) incubation with PD (FIG. 9A).

### Example 24

### PD 404,182 does not Adversely Affect the Growth of Lactobacillus. Lactobacillus

Vaginal microflora is a key component of the innate immune environment and plays an important role in reducing the risk of HIV infection. The dominant bacterial species in healthy woman is Lactobacillus which produces lactic acid, hydrogen peroxide, bacteriocins and other antimicrobial substances that inhibit the growth of pathogenic organisms in the vagina. PD was evaluated for toxicity towards three strains of Lactobacillus normally found in the vagina.

Different bacterial strains were incubated with 300 μM PD or solvent DMSO-containing control media at 37° C. for 30 min and plated on blood agar plates to estimate their corresponding colony forming units per ml (CFU/ml). Less than 1 log difference between control and test CFU is considered non-toxic 17. As shown in Table 2, PD exhibited no adverse effect on all strains of bacteria tested, suggesting that PD is non-toxic to commensal Lactobacillus species.

TABLE 2
CFU of Lactobacillus treated with 300 μM PD.

| Bacterial Strain | Control | PD | Log(Control) − Log(PD) |
|---|---|---|---|
| Lactobacillus | 7,580,000 | 7,260,000 | −0.019 |
| crispatus | | | |
| ATCC 33197 | | | |
| Lactobacillus | 9,986,667 | 7,533,333 | −0.122 |
| jensenii | | | |
| ATCC 25258 | | | |
| Lactobacillus | 10,520,000 | 10,033,333 | −0.021 |
| jensenii | | | |
| LBP 28Ab | | | |

Table 2.
PD 404,182 does not adversely affect the growth of Lactobacillus. Lactobacillus species present in the vagina represent a key component of the vaginal ecosystem and topical microbicides should not adversely affect their growth.

### Example 25

### Efficacy and Toxicity of PD Evaluated Using Primary Cells

Applicants evaluated the cytotoxicity of PD on seven different human cell lines, including human cervical cells TZM-b1 (HeLa). In all cases PD showed minimal cytotoxicity (CC₅₀>300 μM), giving a therapeutic index (CC₅₀/IC₅₀) of >300 for HIV-1. In the present disclosure, freshly activated human PBMCs pooled from multiple donors were infected with eight HIV-1 clinical isolates representing different viral subtypes and tropisms in the presence of different concentrations of PD. The supernatant reverse transcriptase activity was determined 7 days later and used as an indication of HIV infection. The toxicity of PD was determined under identical conditions in the absence of HIV infection. As shown in Table 3, PD exhibited antiviral activity towards all the viral isolates tested, with an average IC₅₀ of 0.55 μM (ranging from 0.14 μM with HIV-1 96USNG31 to 1.18 μM with HIV-1 92UG029). A 48% reduction in cell viability was observed at the highest tested PD concentration (200 μM), resulting in a CC₅₀ of ˜200 μM, indicating that PD is relatively non-toxic to freshly activated human PBMC. The therapeutic index of PD ranges between 170 (for HIV-1 92USNG31) and 1,015 (for HIV-1 RU132).

TABLE 3
Toxicity and potency of PD in PBMCs

| HIV | | Coreceptor | IC₅₀ | IC₉₀ | CC₅₀ | Therapeutic |
|---|---|---|---|---|---|---|
| Isolate | Clade | usage | (μM) | (μM) | (μM) | index |
| 92RW016 | A | R5 | 0.22 | 0.54 | ~200 | 916 |
| 92UG029 | A | X4 | 1.18 | 4.67 | | 170 |
| 92HT599 | B | X4 | 0.55 | 1.92 | | 364 |
| 93BR021 | B | R5 | 0.6 | 4.26 | | 334 |
| 96USNG31 | C | X4/R5 | 0.14 | 1.62 | | 1,425 |
| 98IN022 | C | R5 | 0.4 | 1.48 | | 506 |
| 92UG001 | D | X4/R5 | 1.11 | 1.89 | | 181 |
| RU132 | G | R5 | 0.2 | 0.53 | | 1,015 |

Freshly activated PBMCs (5 × 10⁴ cells/well) were infected with HIV isolates (MOI = 0.1) in the presence of different concentrations of PD. Seven days after infection, supernatants were collected and analyzed for reverse transcriptase activity. Compound toxicity was determined using a MTS assay in the parallel uninfected plates. Error bars represent standard deviation of triplicate samples from one experiment.

To evaluate the toxicity of PD against other primary cells, increasing concentration of PD was incubated with CD4+ T-lymphocytes, macrophages and dendritic cells for up to 14 days. As shown in FIG. 12, minimum toxicity was observed in all three types of primary cells (CC50>300 μM), further pointing to the extremely low cytotoxicity of PD.

### Example 26

### PD Short-Term Stability

Applicants sought to determine the short-term stability of PD under conditions the compound is likely to encounter if used as a microbicide. The environment of the vagina is highly acidic (pH 3.5-4.9) due to the lactic acid produced by the commensal bacteria Lactobacillus. Exposure to seminal fluid (pH 7.2-8) can raise the vaginal pH to 5.8-7.2 for several hours. Thus, Applicants determined the stability of PD under different pHs at 37° C. (FIG. 13A). PD is highly stable in acidic buffer at pH 4 or 6 at 37° C. Since PD targets a non-envelop protein HIV-1 structural component, Applicants used HIV-1 pseudotyped with VSV-G (VSV-Gpp) for these studies because this virus is easy to generate in high titers and can be handled in a biosafety level-2 (BSL-2) environment. Basic pHs of pH 8 or 10 were observed to compromise PD's activity, but only after extended exposure. For example, PD lost ˜50% antiviral activity after incubation in pH 10 buffer for 48 h and lost ˜20% activity when exposed to pH 8 for 48 h at 37° C. In contrast, no activity loss was observed for PD after 24 h exposure to pH 8 buffer and minimal (˜20%) activity loss was seen after 24 h exposure to pH 10. Taken together, these results indicate that PD will likely be stable in highly acidic cervical fluid and should remain active for at least several hours upon contact with seminal fluid.

Since cervical fluid is a complex mixture, Applicants next determined the stability of PD in cervical fluid. As shown in FIG. 13B, no activity loss was observed after PD was incubated in 20% cervical fluid for 24 h at 37° C. These results indicate that once-a-day application of PD should be adequate to provide protection against HIV infection. The ability of PD to retain its antiviral potency at near-neutral pH suggests that PD may also be formulated as a rectal gel.

### Example 27

### PD Long-Term Stability

To evaluate long-term stability, PD was diluted in DPBS buffered at pH 4 or 7 and incubated at 4° C., room temperature or 42° C. An aliquot was taken every week for determination of antiviral activity. As shown in FIG. 14A, PD is extremely stable when stored in pH 4 buffer and retains full antiviral potency even after 8 weeks at 42° C. At pH 7, PD is stable only at room temperature and 4° C. Storage at 42° C. and pH 7 significantly compromised PD activity after 2 weeks.

Applicants also determined the stability of PD formulated in HEC gel at pH 4, as PD is not stable in the presence of HEC gel at pH 7 (data not shown). PD retains full potency at pH 4 in 1.5% HEC gel at 4° C. and RT for at least 4 weeks. However, PD is not stable under same buffer conditions if stored at 42° C. (FIG. 14B) for more than 2 weeks, despite its stability in pH 4 DPBS buffer.

### Example 28

### HIV-1 does not Acquire Resistance to PD after 60 Days

With the high rate of HIV mutation, an ideal microbicide should have a high threshold for viral resistance development. To gauge the ability of HIV-1 to acquire resistance to PD, HIV-1-positive TZM-b1 cells were passaged in the presence of 1, 5 and 10 μM PD for 60 days. No PD-resistant variants could be detected in the course of this experiment (FIG. 15). The inability of HIV-1 to escape PD inactivation further underscores the potential of PD as an HIV-1 microbicide. Applicants chose TZM-b1 cells for the resistance study because these cells can be passaged for an extended period and remain viable for months even in the presence of viral replication. A similar experiment was performed using freshly activated human PBMCs (2 donors) and no emergence of viral resistance was observed (data no shown). However, HIV-1 infected PBMCs were cultured for only 12 days because significant cell death was observed after this period.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

### REFERENCES

1. Birck, M. R., T. P. Holler, and R. W. Woodard. 2000. Identification of a slow tight-binding inhibitor of 3-deoxy-D-manno-octulosonic acid 8-phosphate synthase. Journal of the American Chemical Society 122:9334-9335.
2. Bobardt, M. D., U. Chatterji, L. Schaffer, L. de Witte, and P. A. Gallay. 2010. Syndecan-Fc hybrid molecule as a potent in vitro microbicidal anti-HIV-1 agent. Antimicrob Agents Chemother 54:2753-66.
3. Bobardt, M. D., G. Cheng, L. de Witte, S. Selvarajah, U. Chatterji, B. E. Sanders-Beer, T. B. Geijtenbeek, F. V. Chisari, and P. A. Gallay. 2008. Hepatitis C virus NSSA anchor peptide disrupts human immunodeficiency virus. Proc Natl Acad Sci USA 105:5525-30.
4. Bremer, C. M., C. Bung, N. Kott, M. Hardt, and D. Glebe. 2009. Hepatitis B virus infection is dependent on cholesterol in the viral envelope. Cell Microbiol 11:249-60.
5. Cannon, P. M., N. Kim, S. M. Kingsman, and A. J. Kingsman. 1996. Murine leukemia virus478 based Tat-inducible long terminal repeat replacement vectors: a new system for anti-human immunodeficiency virus gene therapy. J Virol 70:8234-40.
6. CDC. 2006. The Global HIV/AIDS pandemic, 2006. MMWR Morb Mortal Wkly Rep 55:841-4.
7. Cheng, G., A. Montero, P. Gastaminza, C. Whitten-Bauer, S. F. Wieland, M. Isogawa, B. Fredericksen, S. Selvarajah, P. A. Gallay, M. R. Ghadiri, and F. V. Chisari. 2008. A virocidal amphipathic {alpha}-helical peptide that inhibits hepatitis C virus infection in vitro. Proc Natl Acad Sci USA 105:3088-93.
8. Chockalingam, K., R. L. Simeon, C. M. Rice, and Z. Chen. 2010. A cell protection screen reveals potent inhibitors of multiple stages of the hepatitis C virus life cycle. Proc Natl Acad Sci USA 107:3764-3769.
9. Cowan, S., T. Hatziioannou, T. Cunningham, M. A. Muesing, H. G. Gottlinger, and P. D. Bieniasz. 2002. Cellular inhibitors with Fv1-like activity restrict human and simian immunodeficiency virus tropism. Proc Natl Acad Sci USA 99:11914-9.
10. Deming, P., and I. R. McNicholl. 2011. Coinfection with human immunodeficiency virus and hepatitis C virus: challenges and therapeutic advances insights from the society of infectious diseases pharmacists. Pharmacotherapy 31:357-68.
11. Dhawan, D., and K. H. Mayer. 2006. Microbicides to prevent HIV transmission: overcoming obstacles to chemical barrier protection. J Infect Dis 193:36-44.
12. Enserink, M. 2011. Infectious diseases. First specific drugs raise hopes for hepatitis C. Science 332:159-60.
13. Evans, M. J., T. von Hahn, D. M. Tscherne, A. J. Syder, M. Panis, B. Wolk, T. Hatziioannou, J. A. McKeating, P. D. Bieniasz, and C. M. Rice. 2007. Claudin-1 is a hepatitis C virus co500 receptor required for a late step in entry. Nature 446:801-5.
14. Ganesh, L., K. Leung, K. Lore, R. Levin, A. Panet, O, Schwartz, R. A. Koup, and G. J. Nabel. 2004. Infection of specific dendritic cells by CCR5-tropic human immunodeficiency virus type 1 promotes cell-mediated transmission of virus resistant to broadly neutralizing antibodies. J Virol 78:11980-7.
15. Geijtenbeek, T. B., D. S. Kwon, R. Torensma, S. J. van Vliet, G. C. van Duijnhoven, J. Middel, I. L. Cornelissen, H. S, Nottet, V. N. KewalRamani, D. R. Littman, C. G. Figdor, and Y. van Kooyk. 2000. DC-SIGN, a dendritic cell-specific HIV-1-binding protein that enhances trans-infection of T cells. Cell 100:587-97.
16. Gunther-Ausborn, S., A. Praetor, and T. Stegmann. 1995. Inhibition of influenza-induced membrane fusion by lysophosphatidylcholine. J Biol Chem 270:29279-85.
17. Guo, H., X. Pan, R. Mao, X. Zhang, L. Wang, X. Lu, J. Chang, J. T. Guo, S. Passic, F. C. Krebs, B. Wigdahl, T. K. Warren, C. J. Retterer, S. Bavari, X. Xu, A. Cuconati, and T. M. Block. 2010. Alkylated Porphyrins Have Broad Antiviral Activity against Hepadnaviuses, Flaviviruses, Filoviruses and Arenaviruses. Antimicrob Agents Chemother 55:478-86.
18. Henchal, E. A., and J. R. Putnak. 1990. The dengue viruses. Clin Microbiol Rev 3:376-96.
19. Isojima, Y., M. Nakajima, H. Ukai, H. Fujishima, R. G. Yamada, K. H. Masumoto, R. Kiuchi, M. Ishida, M. Ukai-Tadenuma, Y. Minami, R. Kito, K. Nakao, W. Kishimoto, S. H. Yoo, K. Shimomura, T. Takao, A. Takano, T. Kojima, K. Nagai, Y. Sakaki, J. S. Takahashi, and H. R. Ueda. 2009. CKlepsilon/delta-dependent phosphorylation is a temperature-insensitive, period determining process in the mammalian circadian clock. Proc Natl Acad Sci USA 106:15744-9.
20. Jose, J., J. E. Snyder, and R. J. Kuhn. 2009. A structural and functional perspective of alphavirus replication and assembly. Future Microbiol 4:837-56.
21. Kalen, M., E. Wallgard, N. Asker, A. Nasevicius, E. Athley, E. Billgren, J. D. Larson, S. A. Wadman, E. Norseng, K. J. Clark, L. He, L. Karlsson-Lindahl, A. K. Hager, H. Weber, H. Augustin, T. Samuelsson, C. K. Kemmet, C. M. Utesch, J. J. Essner, P. B. Hackett, and M. Hellstrom. 2009. Combination of reverse and chemical genetic screens reveals angiogenesis inhibitors and targets. Chem Biol 16:432-41.
22. Klasse, P. J., R. Shattock, and J. P. Moore. 2008. Antiretroviral drug-based microbicides to prevent HIV-1 sexual transmission. Annu Rev Med 59:455-71.
23. Kolykhalov, A. A., K. Mihalik, S. M. Feinstone, and C. M. Rice. 2000. Hepatitis C virus531 encoded enzymatic activities and conserved RNA elements in the 3′ nontranslated region are essential for virus replication in vivo. J Virol 74:2046-51.
24. Lederman, M. M., R. E. Offord, and O. Hartley. 2006. Microbicides and other topical strategies to prevent vaginal transmission of HIV. Nat Rev Immunol 6:371-82.
25. Li, Y., K. Svehla, N. L. Mathy, G. Voss, J. R. Mascola, and R. Wyatt. 2006. Characterization of antibody responses elicited by human immunodeficiency virus type 1 primary isolate trimeric and monomeric envelope glycoproteins in selected adjuvants. J Virol 80:1414-26.
26. Lindenbach, B. D., M. J. Evans, A. J. Syder, B. Wolk, T. L. Tellinghuisen, C. C. Liu, T. Maruyama, R. O. Hynes, D. R. Burton, J. A. McKeating, and C. M. Rice. 2005. Complete replication of hepatitis C virus in cell culture. Science 309:623-6.
27. Martin, I., and J. M. Ruysschaert. 1995. Lysophosphatidylcholine inhibits vesicles fusion induced by the NH2-terminal extremity of SIV/HIV fusogenic proteins. Biochim Biophys Acta 1240:95-100.
28. Marukian, S., C. T. Jones, L. Andrus, M. J. Evans, K. D. Ritola, E. D. Charles, C. M. Rice, and L. B. Dustin. 2008. Cell culture-produced hepatitis C virus does not infect peripheral blood mononuclear cells. Hepatology 48:1843-50.
29. Morizono, K., G. Bristol, Y. M. Xie, S. K. Kung, and I. S. Chen. 2001. Antibody-directed targeting of retroviral vectors via cell surface antigens. J Virol 75:8016-20.
30. Moss, B. 2006. Poxvirus entry and membrane fusion. Virology 344:48-54.
31. Operskalski, E. A., and A. Kovacs. 2011. HIV/HCV co-infection: pathogenesis, clinical complications, treatment, and new therapeutic technologies. Curr HIV/AIDS Rep 8:12-22.
32. Pietschmann, T., A. Kaul, G. Koutsoudakis, A. Shavinskaya, S. Kallis, E. Steinmann, K. Abid, F. Negro, M. Dreux, F. L. Cosset, and R. Bartenschlager. 2006. Construction and characterization of infectious intragenotypic and intergenotypic hepatitis C virus chimeras. Proc Natl Acad Sci USA 103:7408-13.
33. Pillay, D. 2007. The priorities for antiviral drug resistance surveillance and research. J Antimicrob Chemother 60 Suppl 1:157-8.27
34. Platt, E. J., K. Wehrly, S. E. Kuhmann, B. Chesebro, and D. Kabat. 1998. Effects of CCR5 and CD4 cell surface concentrations on infections by macrophagetropic isolates of human immunodeficiency virus type 1. J Virol 72:2855-64.
35. Rando, R. F., S. Obara, M. C. Osterling, M. Mankowski, S. R. Miller, M. L. Ferguson, F. C. Krebs, B. Wigdahl, M. Labib, and H. Kokubo. 2006. Critical design features of phenylcarboxylate-containing polymer microbicides. Antimicrob Agents Chemother 50:3081-9.
36. Reed, L. J., and H. Muench. 1938. A simple method of estimating fifty percent end-points. Am. J. Hyg. 27:493-497.
37. Reesink, H. W., S. Zeuzem, C. J. Weegink, N. Forestier, A. van Vliet, J. van de Wetering de Rooij, L. McNair, S. Purdy, R. Kauffman, J. Alam, and P. L. Jansen. 2006. Rapid decline of viral RNA in hepatitis C patients treated with VX-950: a phase Ib, placebo-controlled, randomized study. Gastroenterology 131:997-1002.
38. Rice, C. M., R. Levis, J. H. Strauss, and H. V. Huang. 1987. Production of infectious RNA transcripts from Sindbis virus cDNA clones: mapping of lethal mutations, rescue of a temperature-sensitive marker, and in vitro mutagenesis to generate defined mutants. J Virol 61:3809-19.
39. Shattock, R. J., and J. P. Moore. 2003. Inhibiting sexual transmission of HIV-1 infection. Nat Rev Microbiol 1:25-34.
40. Shepard, C. W., L. Finelli, and M. J. Alter. 2005. Global epidemiology of hepatitis C virus infection. Lancet Infect Dis 5:558-67.
41. Soares, M. M., S. W. King, and P. E. Thorpe. 2008. Targeting inside-out phosphatidylserine as a therapeutic strategy for viral diseases. Nat Med 14:1357-62.
42. St Vincent, M. R., C. C. Colpitts, A. V. Ustinov, M. Muqadas, M. A. Joyce, N. L. Barsby, R. F. Epand, R. M. Epand, S. A. Khramyshev, O. A. Valueva, V. A. Korshun, D. L. Tyrrell, and L. M. Schang. 2010. Rigid amphipathic fusion inhibitors, small molecule antiviral compounds against enveloped viruses. Proc Natl Acad Sci USA 107:17339-44.
43. St Vincent, M. R., C. C. Colpitts, A. V. Ustinov, M. Muqadas, M. A. Joyce, N. L. Barsby, R. F. Epand, R. M. Epand, S. A. Khramyshev, O. A. Valueva, V. A. Korshun, D. L. Tyrrell, and L. M. Schang. 2010. Rigid amphipathic fusion inhibitors, small molecule antiviral compounds against enveloped viruses. Proc Natl Acad Sci USA.
44. Susser, S., C. Welsch, Y. Wang, M. Zettler, F. S. Domingues, U. Karey, E. Hughes, R. Ralston, X. Tong, E. Herrmann, S. Zeuzem, and C. Sarrazin. 2009. Characterization of resistance to the protease inhibitor boceprevir in hepatitis C virus-infected patients. Hepatology 50:1709-18.
45. Takeuchi, T., A. Katsume, T. Tanaka, A. Abe, K. Inoue, K. Tsukiyama-Kohara, R. Kawaguchi, S. Tanaka, and M. Kohara. 1999. Real-time detection system for quantification of hepatitis C virus genome. Gastroenterology 116:636-42.
46. UNAIDS. 2010. UNAIDS Report on the Global AIDS Epidemic 2010.
47. Welsch, S., S. Miller, I. Romero-Brey, A. Merz, C. K. Bleck, P. Walther, S. D. Fuller, C. Antony, J. Krijnse-Locker, and R. Bartenschlager. 2009. Composition and three-dimensional architecture of the dengue virus replication and assembly sites. CellHost Microbe 5:365-75.
48. Wolf, M. C., A. N. Freiberg, T. Zhang, Z. Akyol-Ataman, A. Grock, P. W. Hong, J. Li, N. F. Watson, A. Q. Fang, H. C. Aguilar, M. Porotto, A. N. Honko, R. Damoiseaux, J. P. Miller, S. E. Woodson, S. Chantasirivisal, V. Fontanes, O. A. Negrete, P. Krogstad, A. Dasgupta, A. Moscona, L. E. Hensley, S. P. Whelan, K. F. Faull, M. R. Holbrook, M. E. Jung, and B. Lee. 2010. A broad-spectrum antiviral targeting entry of enveloped viruses. Proc Natl Acad Sci USA 107:3157-3162.
49. Zennou, V., and P. D. Bieniasz. 2006. Comparative analysis of the antiretroviral activity of APOBEC3G and APOBEC3F from primates. Virology 349:31-40.
50. Henry S D, van der Wegen P, Metselaar H J, Tilanus H W, Scholte B J, et al. (2006) Simultaneous targeting of HCV replication and viral binding with a single lentiviral vector containing multiple RNA interference expression cassettes. Mol Ther 14: 485-493.
51. Yeni P G, Hammer S M, Hirsch M S, Saag M S, Schechter M, et al. (2004) Treatment for adult HIV infection: 2004 recommendations of the International AIDS Society-USA Panel. JAMA 292: 251-265.
52. Naswa S, Marfatia Y S, Prasad T L (2012) Microbicides and HIV: A Review and an update. Indian J Sex Transm Dis 33: 81-90.
53. Nuttall J, Romano J, Douville K, Galbreath C, Nel A, et al. (2007) The future of HIV prevention: prospects for an effective anti-HIV microbicide. Infect Dis Clin North Am 21: 219-239, x.
54. Youle M, Wainberg M A (2003) Pre-exposure chemoprophylaxis (PREP) as an HIV prevention strategy. Journal of the International Association of Physicians in AIDS Care 2: 102-105.
55. Olsen J S, Easterhoff D, Dewhurst S (2011) Advances in HIV microbicide development. Future medicinal chemistry 3: 2101-2116.
56. Doncel G F, Clark M R (2010) Preclinical evaluation of anti-HIV microbicide products: New models and biomarkers. Antiviral Res 88 Suppl 1: S10-18.
57. Desai M, Iyer G, Dikshit R K (2012) Antiretroviral drugs: critical issues and recent advances. Indian J Pharmacol 44: 288-298.
58. Vanpouille C, Arakelyan A, Margolis L (2012) Microbicides: still a long road to success. Trends Microbiol 20: 369-375.
59. Pozzetto B, Delezay O, Brunon-Gagneux A, Hamzeh-Cognasse H, Lucht F, et al. (2012) Current and future microbicide approaches aimed at preventing HIV infection in women. Expert Rev Anti Infect Ther 10: 167-183.
60. Abdool Karim Q, Abdool Karim S S, Frohlich J A, Grobler A C, Baxter C, et al. (2010) Effectiveness and safety of tenofovir gel, an antiretroviral microbicide, for the prevention of HIV infection in women. Science 329: 1168-1174.
61. Holmes D (2012) FDA paves the way for pre-exposure HIV prophylaxis. Lancet 380: 325.
62. Olsen J S, Easterhoff D, Dewhurst S (2011) Advances in HIV microbicide development. Future Med Chem 3: 2101-2116.
63. Chamoun A M, Chockalingam K, Bobardt M, Simeon R, Chang J, et al. (2012) PD 404,182 Is a Virocidal Small Molecule That Disrupts Hepatitis C Virus and Human Immunodeficiency Virus. Antimicrobial agents and chemotherapy 56: 672-681.
64. Rosenberg Z (2011) Current advances in microbicides. Tropical Medicine & International Health 16: 14-15.
65. Byeon U, Louis J M, Gronenborn A M (2003) A protein contortionist: core mutations of GB 1 that induce dimerization and domain swapping. Journal of Molecular Biology 333: 141-152.
66. Geijtenbeek T B, Kwon D S, Torensma R, van Vliet S J, van Duijnhoven G C, et al. (2000) DC-SIGN, a dendritic cell-specific HIV-1-binding protein that enhances trans-infection of T cells. Cell 100: 587-597.
67. Mizuhara T, Oishi S, Fujii N, Ohno H (2010) Efficient synthesis of pyrimido[1,2-c][1,3]benzothiazin-6-imines and related tricyclic heterocycles by S(N)Ar-type C—S, C—N, or C—O bond formation with heterocumulenes. J Org Chem 75: 265-268.
68. Introini A, Vanpouille C, Lisco A, Grivel J C, Margolis L (2013) Interleukin-7 Facilitates HIV-1 Transmission to Cervico-Vaginal Tissue ex vivo. PLoS Pathog 9: e1003148.
69. Munch J, Rucker E, Standker L, Adermann K, Goffinet C, et al. (2007) Semen-derived amyloid fibrils drastically enhance HIV infection. Cell 131: 1059-1071.
70. Neurath A R, Strick N, Li Y Y (2006) Role of seminal plasma in the anti-HIV-1 activity of candidate microbicides. BMC Infect Dis 6: 150.
71. Karim S S A (2010) Results of effectiveness trials of PRO 2000 gel: lessons for future microbicide trials. Future Microbiology 5: 527-529.
72. Patel S, Hazrati E, Cheshenko N, Galen B, Yang H, et al. (2007) Seminal plasma reduces the effectiveness of topical polyanionic microbicides. J Infect Dis 196: 1394-1402.
73. Platt E J, Bilska M, Kozak S L, Kabat D, Montefiori D C (2009) Evidence that Ecotropic Murine Leukemia Virus Contamination in TZM-b1 Cells Does Not Affect the Outcome of Neutralizing Antibody Assays with Human Immunodeficiency Virus Type 1. Journal of Virology 83: 8289-8292.
74. Takeuchi Y, McClure M O, Pizzato M (2008) Identification of Gammaretroviruses Constitutively Released from Cell Lines Used for Human Immunodeficiency Virus Research. Journal of Virology 82: 12585-12588.
75. Wei X P, Decker J M, Liu H M, Zhang Z, Arani R B, et al. (2002) Emergence of resistant human immunodeficiency virus type 1 in patients receiving fusion inhibitor (T-20) monotherapy. Antimicrobial Agents and Chemotherapy 46: 1896-1905.
76. Derdeyn C A, Decker J M, Sfakianos J N, Wu X, O'Brien W A, et al. (2000) Sensitivity of human immunodeficiency virus type 1 to the fusion inhibitor T-20 is modulated by coreceptor specificity defined by the V3 loop of gp120. J Virol 74: 8358-8367.
77. Platt E J, Wehrly K, Kuhmann S E, Chesebro B, Kabat D (1998) Effects of CCR5 and CD4 cell surface concentrations on infections by macrophagetropic isolates of human immunodeficiency virus type 1. J Virol 72: 2855-2864.
78. Kempf C, Jentsch P, Barre-Sinoussi F B, Poirier B, Morgenthaler J J, et al. (1991) Inactivation of human immunodeficiency virus (HIV) by low pH and pepsin. J Acquir Immune Defic Syndr 4: 828-830.
79. Klebanoff S J, Coombs R W (1991) Viricidal effect of Lactobacillus acidophilus on human immunodeficiency virus type 1: possible role in heterosexual transmission. J Exp Med 174: 289-292.
80. Hawes S E, Hillier S L, Benedetti J, Stevens C E, Koutsky L A, et al. (1996) Hydrogen peroxide-producing lactobacilli and acquisition of vaginal infections. J Infect Dis 174: 1058-1063.
89. Zheng H Y, Alcorn T M, Cohen M S (1994) Effects of H2O2-producing lactobacilli on Neisseria gonorrhoeae growth and catalase activity. J Infect Dis 170: 1209-1215.
90. Pavlova S I, Kilic A O, Kilic S S, So J S, Nader-Macias M E, et al. (2002) Genetic diversity of vaginal lactobacilli from women in different countries based on 16S rRNA gene sequences. J Appl Microbiol 92: 451-459.
91. Moncla B J, Pryke K, Rohan L C, Yang H (2012) Testing of viscous anti-HIV microbicides using Lactobacillus. J Microbiol Methods 88: 292-296.
92. Owen D H, Katz D F (1999) A vaginal fluid simulant. Contraception 59: 91-95.
93. Owen D H, Katz D F (2005) A review of the physical and chemical properties of human semen and the formulation of a semen simulant. J Androl 26: 459-469.
94. Fox C A, Meldrum S J, Watson B W (1973) Continuous measurement by radio-telemetry of vaginal pH during human coitus. J Reprod Fertil 33: 69-75.
95. Mesquita P M, Cheshenko N, Wilson S S, Mhatre M, Guzman E, et al. (2009) Disruption of tight junctions by cellulose sulfate facilitates HIV infection: model of microbicide safety. J Infect Dis 200: 599-608.
96. Hoffman I F, Taha T E, Padian N S, Kelly C W, Welch J D, et al. (2004) Nonoxynol-9 100 mg gel: multi-site safety study from sub-Saharan Africa. AIDS 18: 2191-2195.
97. Veazey R S, Ketas T J, Dufour J, Moroney-Rasmussen T, Green L C, et al. (2010) Protection of rhesus macaques from vaginal infection by vaginally delivered maraviroc, an inhibitor of HIV-1 entry via the CCR5 co-receptor. J Infect Dis 202: 739-744.
98. Lederman M M, Veazey R S, Offord R, Mosier D E, Dufour J, et al. (2004) Prevention of vaginal SHIV transmission in rhesus macaques through inhibition of CCR5. Science 306: 485-487.
99. Neff C P, Kurisu T, Ndolo T, Fox K, Akkina R (2011) A topical microbicide gel formulation of CCR5 antagonist maraviroc prevents HIV-1 vaginal transmission in humanized RAG-hu mice. PLoS One 6: e20209.
100. Veazey R S, Klasse P J, Schader S M, Hu Q, Ketas T J, et al. (2005) Protection of macaques from vaginal SHIV challenge by vaginally delivered inhibitors of virus-cell fusion. Nature 438: 99-102.
101. Tsai C C, Emau P, Jiang Y, Tian B, Morton W R, et al. (2003) Cyanovirin-N gel as a topical microbicide prevents rectal transmission of SHIV89.6P in macaques. AIDS Res Hum Retroviruses 19: 535-541.

What is claimed is:

1. A method of inactivating a RNA virus of the family retroviridae in a biological sample, the method comprising: contacting the biological sample with an effective amount of an antiviral composition comprising PD 404,182.

2. The method of claim 1, wherein the RNA virus is selected from the group consisting of HIV pseudotyped lentiviruses, primary human immunodeficiency virus-1 isolates (HIV-1), human immunodeficiency virus-2 (HIV-2), and simian immunodeficiency virus (SIV).

3. The method of claim 1, wherein the inactivation of the virus particle by PD 404,182 is mediated by physical disruption of the virion.

4. The method of claim 1, wherein the virus particle in the biological sample is associated with a cell.

5. The method of claim 1, wherein the virus particle in the biological sample is cell-free.

6. The method of claim 1, wherein the inactivation of the virus particle occurs at a temperature range of about 25° C. to about 37° C.

7. The method of claim 1, wherein the biological sample is selected from the group consisting of: blood, a blood product, cells, a tissue, an organ, sperm, a vaccine formulation, and a bodily fluid.

8. The method of claim 7, wherein the blood is from a blood transfusion.

9. A method of treating a subject infected by a RNA virus of the family retroviridae comprising:
administering to the subject a therapeutically or prophylactically effective amount of an antiviral composition comprising PD 404,182 or a pharmaceutically acceptable salt thereof.

10. The method of claim 9, wherein the subject is a human.

11. The method of claim 9, wherein the RNA virus of the retroviridae family is selected from the group consisting of HIV pseudotyped lentiviruses, human immunodeficiency virus-1 (HIV-1), human immunodeficiency virus-2 (HIV-2), and simian immunodeficiency virus (SIV).

12. The method of claim 9, wherein the antiviral activity of PD 404,182 is independent of viral envelope proteins.

13. The method of claim 9, wherein the RNA virus of the reteroviridae family is the human Immunodeficiency Virus-1 (HIV-1) or the Human Immunodeficiency virus-2 (HIV-2).

15. The method of claim 9, wherein the antiviral activity of PD 404,182 is mediated by inactivation of extracellular virions.

16. The method of claim 9, wherein the antiviral action of PD 404,182 is mediated by physical disruption of the virion.

17. The method of claim 13, wherein the subject has a Hepatitis C Virus (HCV) co-infection.

18. The method of claim 17, wherein the method further comprises administering to the subject one or more additional therapeutic agents.

19. The method of claim 18, wherein one or more additional therapeutic agents are selected from the group consisting of interferon agent, ribavirin, HCV inhibitor, and HIV inhibitor.

20. The method of claim 9, wherein the antiviral composition is administered intravenously, orally, subcutaneously, intramuscularly or transdermally.

21. The method of claim 9, wherein the antiviral composition is administered transdermally.

22. The method of claim 21, wherein the antiviral composition is in the form of a gel, foam, cream, ointment, lotion, balm, wax, salve, solution, condom coated with the composition, suppository, suspension and spray.

23. A method of preventing transmission of infection of a RNA virus of the family retroviridae, to a subject in need thereof comprising: contacting a mucus membrane of the subject with a topical formulation comprising an effective amount of PD 404,182 or a pharmaceutically acceptable salt thereof, wherein the viral infection is caused by a retrovirus.

24. The method of claim 23, wherein the formulation is in the form of a gel, foam, cream, ointment, lotion, balm, wax, salve, solution, suppository, condom coated with the composition, suspension and spray.

25. The method of claim 23 wherein the mucus membrane is the mucus membrane of the cervix or of the rectum.

26. The method of claim 23, wherein the RNA virus of the reteroviridae family is selected from the group consisting of HIV pseudotyped lentiviruses, human immunodeficiency virus-1 (HIV-1), human immunodeficiency virus-2 (HIV-2), and simian immunodeficiency virus (SIV).

27. The method of claim 23, wherein the RNA virus of the reteroviridae family is the human immunodeficiency virus-1 (HIV-1) or the human immunodeficiency virus-2 (HIV-2).

28. The method of claim 27, wherein the subject has a Hepatitis C Virus (HCV) co-infection.

29. The method of claim 23, wherein the topical formulation exhibits low cytotoxicity.
